(19) United States
(12) Patent Application Publication
(10) Pub. No.: US 2021/0393707 A1
(43) Pub. Date: Dec. 23, 2021

(54) SYSTEMS AND METHODS FOR TREATING A DYSBIOSIS USING FECAL-DERIVED BACTERIAL POPULATIONS

(71) Applicant: NUBIYOTA LLC, Teaneck, NJ (US)

(72) Inventor: Emma ALLEN-VERCOE, Guelph (CA)

(21) Appl. No.: 17/348,011

(22) Filed: Jun. 15, 2021

Related U.S. Application Data
(63) Continuation of application No. 15/754,765, filed on Feb. 23, 2018, now abandoned.
Application No. 15/754,765 is a continuation of application No. PCT/US2016/048312, filed on Aug. 24, 2016.
(60) Provisional application No. 62/209,149, filed on Aug. 24, 2015.

(51) Int. Cl. A61K 35/742; A61P 1/04

(52) U.S. Cl. CPC A61K 35/742; A61P 1/04

(57) ABSTRACT

The present invention provides a method, wherein the method treats a subject having a dysbiosis, the method comprising: determining a first metabolic profile of the gut microbiome of a subject having a dysbiosis; changing the first metabolic profile of the gut microbiome of the subject to a second metabolic profile of the gut microbiome of the subject, by administering to the subject a composition comprising at least one bacterial species selected from the group consisting of: Acidaminococcus intestinalis, Bacteroides ovatus, Bifidobacterium adolescentis, Bifidobacterium longum, Blautia sp., Clostridium sp., Collinsella aerofaciens, Escherichia coli, Eubacterium desmolans, Eubacterium eligens, Eubacterium limosum, Faecalibacterum prausnitzii, Lachnospira pectinoschiza, Lactobacillus casei, Parabacteroides distasonis, Roseburia faecalis, Roseburia intestinalis, Ruminococcus sp., Ruminococcus species, and Ruminococcus torques, wherein the composition is administered at a therapeutically effective amount, sufficient to alter the first metabolic profile of the gut microbiome to the second metabolic profile of the gut microbiome.

### RELATED APPLICATIONS

This application claims the priority of U.S. provisional application U.S. Patent Application No. 62/209,149; filed Aug. 24, 2015; entitled “OPTIMIZING STOOL SUBSTITUTE TRANSPLANT THERAPY FOR THE ERADICATION OF CLOSTRIDIUM DIFFICILE INFECTION USING WHOLE GENOME ANALYSIS,” which is incorporated herein by reference in its entirety for all purposes.

### FIELD OF THE INVENTION

The field of the invention relates to therapies for treating gastrointestinal disorders. In particular, the present invention provides systems and methods for characterizing compositions comprising fecal-derived bacterial populations used as therapies for treating gastrointestinal disorders.

### BACKGROUND OF THE INVENTION

Clostridium difficile is a toxin-producing, Gram-positive bacillus whose overabundance in the human gut leads to the production of toxins and the colitis symptoms of Clostridium difficile infection (CDI). CDI is an opportunistic bacterial disease of the gastrointestinal tract, which accounts for 15-25% of all antibiotic-associated diarrhea cases. The increased use of broad-spectrum systemic antimicrobials, which disrupt the ecological bacterial balance of the human gut, has made CDI a growing complication in the medical field.

CDI is treated with metronidazole or oral vancomycin for 10-14 days. However, between 5% and 35% of patients who receive treatment relapse. Recurrent CDI (RCDI) is defined as complete resolution of CDI while on appropriate therapy followed by recurrence of infection after treatment has been stopped. It is widely believed in the medical community that RCDI is not necessarily caused by the pathogen itself, but by an inability to re-establish normal intestinal bacteria.

Compositions comprising fecal-derived bacterial populations may be used to treat CDI, as well as other causes resulting in dysbiosis.

### BRIEF DESCRIPTION OF THE FIGURES

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

FIGS. 1A-F shows sequence comparisons employed in the methods according to some embodiments of the present invention.

FIGS. 2A-F shows sequence alignment diagrams employed in the methods according to some embodiments of the present invention.

FIGS. 3A-C shows some scatter plots used for comparisons employed in the methods according to some embodiments of the present invention.

FIGS. 4A-D shows some comparisons for identifying species matches employed in the methods according to some embodiments of the present invention.

FIGS. 5A-5H show KEGG pathway maps used to identify metabolic pathways employed in the methods according to some embodiments of the present invention.

FIGS. 6A-6H show a metabolic pathway map of one or more species employed in the methods according to some embodiments of the present invention.

FIGS. 7A-7Q show metabolic pathway maps employed in the methods according to some embodiments of the present invention.

FIGS. 8A-8H show a pathway map to compare 22 species employed in the methods according to some embodiments of the present invention.

FIGS. 9 and 10 show a single-stage chemostat vessel employed in the methods according to some embodiments of the present invention.

### SUMMARY OF INVENTION

In some embodiments, the present invention provides a method, wherein the method treats a subject having a dysbiosis, the method comprising: determining a first metabolic profile of the gut microbiome of a subject having a dysbiosis; changing the first metabolic profile of the gut microbiome of the subject to a second metabolic profile of the gut microbiome of the subject, by administering to the subject a composition comprising at least one bacterial strain selected from the group consisting of: Acidaminococcus intestinalis 14LG, Bacteroides ovatus 5MM Bifidobacterium adolescentis 20MRS, Bifidobacterium longum, Blautia sp. 27FM, Clostridium sp. 21FAA, Collinsella aerofaciens, Escherichia coli 3FM4i, Eubacterium desmolans 48FAA, Eubacterium eligens F1FAA, Eubacterium limosum 13LG, Faecalibacterium prausnitzii 40FAA, Lachnospira pectinoschiza 34FAA, Lactobacillus casei 25MRS, Parabacteroides distasonis 5FM, Roseburia faecalis 39FAA, Roseburia intestinalis 31FAA, Ruminococcus sp. 11FM, Ruminococcus species, and Ruminococcus torques 30FAA, wherein the composition is administered at a therapeutically effective amount, sufficient to alter the first metabolic profile of the gut microbiome to the second metabolic profile of the gut microbiome, wherein the first metabolic profile of the gut microbiome is a consequence of the dysbiosis, wherein the second metabolic profile of the gut microbiome treats the subject having the dysbiosis.

In some embodiments, the composition is administered at a therapeutically effective amount, sufficient to colonize the gut of the subject.

In some embodiments, the composition comprises at least one bacterial strain selected from the group consisting of: 16-6-I 21 FAA 92% Clostridium cocleatum; 16-6-I 2 MRS 95% Blautia luti; 16-6-134 FAA 95% Lachnospira pectinoschiza; 32-6-130 D6 FAA 96% Clostridium glycyrrhizinilyticum; and 32-6-I 28 D6 FAA 94% Clostridium lactatifermentans.

In some embodiments, the present invention provides a method, wherein the method treats a subject having a dysbiosis, the method comprising: determining a first metabolic profile of the gut microbiome of a subject having a dysbiosis; changing the first metabolic profile of the gut microbiome of the subject to a second metabolic profile of the gut microbiome of the subject, by administering to the subject a composition comprising at least one bacterial species selected from the group consisting of: Acidaminococcus intestinalis, Bacteroides ovatus, Bifidobacterium adolescentis, Bifidobacterium longum, Blautia sp., Clostridium sp., Collinsella aerofaciens, Escherichia coli, Eubacterium desmolans, Eubacterium eligens, Eubacterium limosum, Faecalibacterium prausnitzii, Lachnospira pectinoschiza, Lactobacillus casei, Parabacteroides distasonis, Roseburia faecalis, Roseburia intestinalis, Ruminococcus sp., Ruminococcus species, and Ruminococcus torques, wherein the composition is administered at a therapeutically effective amount, sufficient to alter the first metabolic profile of the gut microbiome to the second metabolic profile of the gut microbiome, wherein the first metabolic profile of the gut microbiome is a consequence of the dysbiosis, wherein the second metabolic profile of the gut microbiome treats the subject having the dysbiosis.

In some embodiments, the composition is administered at a therapeutically effective amount, sufficient to colonize the gut of the subject.

In some embodiments, the composition comprises at least one bacterial species selected from the group consisting of: Clostridium cocleatum; Blautia luti; Lachnospira pectinoschiza; Clostridium glycyrrhizinilyticum; and Clostridium lactatifermentans.

In some embodiments, the dysbiosis is associated with gastrointestinal inflammation. In some embodiments, the gastrointestinal inflammation is an inflammatory bowel disease, irritable bowel syndrome, diverticular disease, ulcerative colitis, Crohn's disease, or indeterminate colitis.

In some embodiments, the dysbiosis is a Clostridium difficile infection. In some embodiments, the dysbiosis is food poisoning. In some embodiments, the dysbiosis is chemotherapy-related dysbiosis.

### DETAILED DESCRIPTION OF THE INVENTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the description, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “dysbiosis” refers to an imbalance of a subject's gut microbiome.

As used herein, the term “microbiome” refers to all the microbes in a community. As a non-limiting example, the human gut microbiome includes all of the microbes in the human's gut.

As used herein, the term “chemotherapy-related dysbiosis” refers to any intervention used to target a subject's particular disease which leads to an imbalance of the subject's gut microbiome.

As used herein, the term “fecal bacteriotherapy” refers to a treatment in which donor stool is infused into the intestine of the recipient to re-establish normal bacterial microbiota. Fecal bacteriotherapy has shown promising results in preliminary studies with close to a 90% success rate in 100 patient cases published thus far. Without being bound by theory, it is believed to work through breaking the cycle of repetitive antibiotic use, re-establishing a balanced ecosystem that represses the growth of C. difficile.

As used herein, the term “keystone species” are species of bacteria which are consistently found in human stool samples.

As used herein, the term “OTU” refers to an operational taxonomic unit, defining a species, or a group of species via similarities in nucleic acid sequences, including, but not limited to 16S rRNA sequences.

### Fecal-Derived Bacterial Populations

In some embodiments, the present invention provides a method, wherein the method treats a subject having a dysbiosis, the method comprising: determining a first metabolic profile of the gut microbiome of a subject having a dysbiosis; changing the first metabolic profile of the gut microbiome of the subject to a second metabolic profile of the gut microbiome of the subject, by administering to the subject a composition comprising at least one bacterial strain selected from the group consisting of: Acidaminococcus intestinalis 14LG, Bacteroides ovatus 5MM Bifidobacterium adolescentis 20MRS, Bifidobacterium longum, Blautia sp. 27FM, Clostridium sp. 21FAA, Collinsella aerofaciens, Escherichia coli 3FM4i, Eubacterium desmolans 48FAA, Eubacterium eligens F1FAA, Eubacterium limosum 13LG, Faecalibacterium prausnitzii 40FAA, Lachnospira pectinoschiza 34FAA, Lactobacillus casei 25MRS, Parabacteroides distasonis 5FM, Roseburia faecalis 39FAA, Roseburia intestinalis 31FAA, Ruminococcus sp. 11FM, Ruminococcus species, and Ruminococcus torques 30FAA, wherein the composition is administered at a therapeutically effective amount, sufficient to alter the first metabolic profile of the gut microbiome to the second metabolic profile of the gut microbiome, wherein the first metabolic profile of the gut microbiome is a consequence of the dysbiosis, wherein the second metabolic profile of the gut microbiome treats the subject having the dysbiosis.

In some embodiments, the composition is administered at a therapeutically effective amount, sufficient to colonize the gut of the subject.

In some embodiments, the composition comprises at least one bacterial strain selected from the group consisting of: 16-6-I 21 FAA 92% Clostridium cocleatum; 16-6-I 2 MRS 95% Blautia luti; 16-6-134 FAA 95% Lachnospira pectinoschiza; 32-6-130 D6 FAA 96% Clostridium glycyrrhizinilyticum; and 32-6-I 28 D6 FAA 94% Clostridium lactatifermentans.

In some embodiments, the present invention provides a method, wherein the method treats a subject having a dysbiosis, the method comprising: determining a first metabolic profile of the gut microbiome of a subject having a dysbiosis; changing the first metabolic profile of the gut microbiome of the subject to a second metabolic profile of the gut microbiome of the subject, by administering to the subject a composition comprising at least one bacterial species selected from the group consisting of: Acidaminococcus intestinalis, Bacteroides ovatus, Bifidobacterium adolescentis, Bifidobacterium longum, Blautia sp., Clostridium sp., Collinsella aerofaciens, Escherichia coli, Eubacterium desmolans, Eubacterium eligens, Eubacterium limosum, Faecalibacterium prausnitzii, Lachnospira pectinoschiza, Lactobacillus casei, Parabacteroides distasonis, Roseburia faecalis, Roseburia intestinalis, Ruminococcus sp., Ruminococcus species, and Ruminococcus torques, wherein the composition is administered at a therapeutically effective amount, sufficient to alter the first metabolic profile of the gut microbiome to the second metabolic profile of the gut microbiome, wherein the first metabolic profile of the gut microbiome is a consequence of the dysbiosis, wherein the second metabolic profile of the gut microbiome treats the subject having the dysbiosis.

In some embodiments, the composition is administered at a therapeutically effective amount, sufficient to colonize the gut of the subject.

In some embodiments, the composition comprises at least one bacterial species selected from the group consisting of: Clostridium cocleatum; Blautia luti; Lachnospira pectinoschiza; Clostridium glycyrrhizinilyticum; and Clostridium lactatifermentans.

In some embodiments, the dysbiosis is associated with gastrointestinal inflammation. In some embodiments, the gastrointestinal inflammation is an inflammatory bowel disease, irritable bowel syndrome, diverticular disease, ulcerative colitis, Crohn's disease, or indeterminate colitis.

In some embodiments, the dysbiosis is a Clostridium difficile infection. In some embodiments, the dysbiosis is food poisoning. In some embodiments, the dysbiosis is chemotherapy-related dysbiosis.

In some embodiments, at least one bacterial species is disclosed in ‘Stool substitute transplant therapy for the eradication of Clostridium difficile infection: ‘RePOOPulating the gut’, by Petrof et al. (2013), which is incorporated herein by reference in its entirety.

In some embodiments, at least one bacterial species is disclosed in Kurokawa et al., “Comparative metagenomics revealed commonly enriched gene sets in human gut microbiomes”, (2007) DNA Research 14: 169-181, which is incorporated herein by reference in its entirety.

In some embodiments, the at least one bacterial species is disclosed in U.S. Patent Application Publication No. 20150044173. Alternatively, in some embodiments, the at least one bacterial species is disclosed in U.S. Patent Application No. 20140363397. Alternatively, in some embodiments, the at least one bacterial species is disclosed in U.S. Patent Application No. 20140086877. Alternatively, in some embodiments, the at least one bacterial species is disclosed in U.S. Pat. No. 8,906,668.

In some embodiments, the method of the present invention can include evaluating at least one bacteria according to the disclosed methods in Takagi et al. (2016) “A single-batch fermentation system to simulate human colonic microbiota for high-throughput evaluation of prebiotics” PLoS ONE 11(8): e0160533.

In some embodiments, the at least one bacterial species is derived from a healthy patient. In some embodiments, the at least one bacterial species is derived from a healthy patient according to the methods disclosed in U.S. Patent Application Publication No. 20140342438.

In some embodiments, the at least one bacterial species and/or strain is derived from a patient by a method comprising:
a. obtaining a freshly voided stool sample, and placing the sample in an anaerobic chamber (in an atmosphere of 90% N2, 5% CO2, and 5% H2);
b. generating a fecal slurry by macerating the stool sample in a buffer; and
c. removing food particles by centrifugation, and retaining the supernatant.

In some embodiments, the supernatant is used to seed a chemostat according to the methods of U.S. Publication Number 20140342438.

Culture Methods According to Some Embodiments of the Present Invention

The effectiveness of the method to determine a first metabolic profile of the gut microbiome of a subject having a dysbiosis can be limited by factors such as, for example, the sensitivity of the method (i.e., the method is only capable of detecting a particular bacterial strain if the strain is present above a threshold level.)

The effectiveness of the method to determine a second metabolic profile of the gut microbiome can be limited by factors such as, for example, the sensitivity of the method (i.e., the method is only capable of detecting a particular bacterial strain if the strain is present above a threshold level.)

In some embodiments, the threshold level is dependent on the sensitivity of the detection method. Thus, in some embodiments, depending on the sensitivity of the detection method, a greater amount of the at least one bacterial species is required to determine if there has been sufficient colonization of the subject.

In some embodiments, the at least one bacterial strain is cultured in a chemostat vessel. In some embodiments, the at least one bacterial strain is selected from the group consisting of: Acidaminococcus intestinalis 14LG, Bacteroides ovatus 5A/P14, Bifidobacterium adolescentis 20MRS, Bifidobacterium longum, Blautia sp. 27FM, Clostridium sp. 21FAA, Collinsella aerofaciens, Escherichia coli 3FM4i, Eubacterium desmolans 48FAA, Eubacterium eligens F1FAA, Eubacterium limosum 13LG, Faecalibacterium prausnitzii 40FAA, Lachnospira pectinoschiza 34FAA, Lactobacillus casei 25MRS, Parabacteroides distasonis 5FM, Roseburia faecalis 39FAA, Roseburia intestinalis 31FAA, Ruminococcus sp. 11FM, Ruminococcus species, Ruminococcus torques 30FAA; and any combination thereof, is cultured in a chemostat vessel.

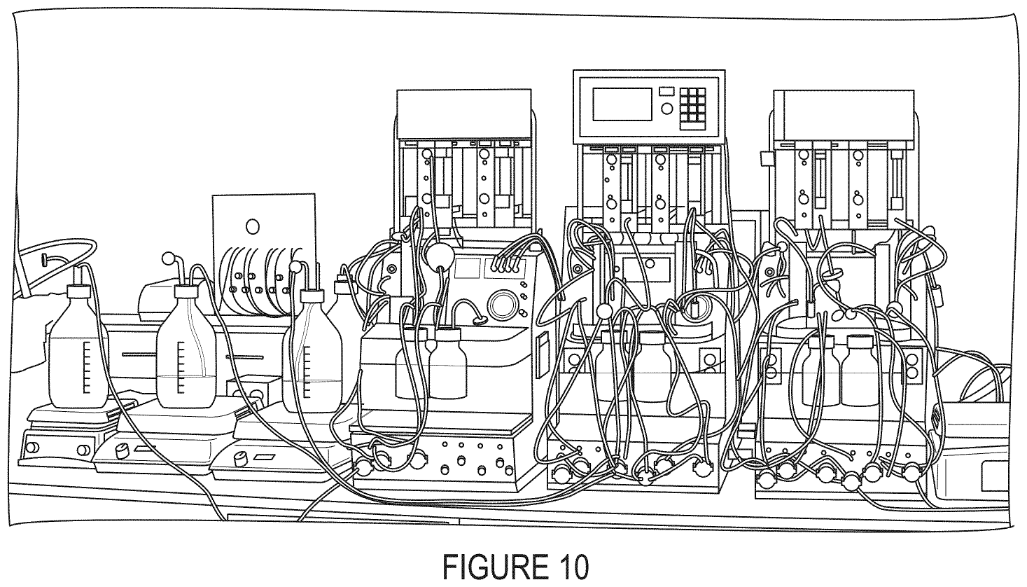

In some embodiments, the at least one bacterial strain is selected from the group consisting of: 16-6-I 21 FAA 92% Clostridium cocleatum; 16-6-I 2 MRS 95% Blautia luti; 16-6-I 34 FAA 95% Lachnospira pectinoschiza; 32-6-I 30 D6 FAA 96% Clostridium glycyrrhizinilyticum; 32-6-I 28 D6 FAA 94% Clostridium lactatifermentans; and any combination thereof, is cultured in a chemostat vessel. In some embodiments, the chemostat vessel is the vessel disclosed in U.S. Patent Application Publication No. 20140342438. In an embodiment, the chemostat vessel is the vessel described in FIGS. 9 and 10.

In some embodiments, the chemostat vessel was converted from a fermentation system to a chemostat by blocking off the condenser and bubbling nitrogen gas through the culture. In some embodiments, the pressure forces the waste out of a metal tube (formerly a sampling tube) at a set height and allows for the maintenance of given working volume of the chemostat culture.

In some embodiments, the chemostat vessel is kept anaerobic by bubbling filtered nitrogen gas through the chemostat vessel. In some embodiments, temperature and pressure are automatically controlled and maintained.

In some embodiments, the culture pH of the chemostat culture is maintained using 5% (v/v) HCl (Sigma) and 5% (w/v) NaOH (Sigma).

In some embodiments, the culture medium of the chemostat vessel is continually replaced. In some embodiments, the replacement occurs over a period of time equal to the retention time of the distal gut. Consequently, in some embodiments, the culture medium is continuously fed into the chemostat vessel at a rate of 400 mL/day (16.7 mL/hour) to give a retention time of 24 hours, a value set to mimic the retention time of the distal gut. An alternate retention time can be 65 hours (approximately 148 mL/day, 6.2 mL/hour). In some embodiments, the retention time can be as short as 12 hours.

In some embodiments, the culture medium is a culture medium disclosed in U.S. Patent Application Publication No. 20140342438.

### Materials and Methods

Genome Sequences

The data for this study includes the draft genome sequences (in contig form) of thirty-three bacteria strains, which are disclosed in Table 4. The bacterial genomes were sequenced using the Illumina MiSeq Platform. Species were named according to closest match by comparison of full-length 16S rRNA genes and may not reflect the true speciation of the bacteria, for simplicity bacteria used in Part I have been given a separate identity as strain A or strain B, Table 1 provides the true identification for these strains.

Study Design

The study includes three stages. The first stage focused on comparing the genomes of species for which pairs of strains had been included in the RePOOPulate study (Petrof et al.) (also referred to as the “original RePOOPulate protoype” or “original RePOOPulate ecosystem”). The genomes of six pairs of species strains that matched closely by full-length 16S sequence alignment were compared in order to search for redundancies. Multiple strains of these bacteria were originally chosen for inclusion in the RePOOPulate ecosystem based on morphological and behavioral differences in the cultured bacteria. The goal of this portion of the project was to determine whether the use of multiple strains was redundant or if there is a true genetic difference that validates a biologically necessity to include both strains for the maintenance of ecological balance.

The second stage of the project focused on developing a broad pipeline for determining the genetic coverage of the KEGG pathways. KEGG, which stands for Kyoto Encyclopedia of Genes and Genomes, is a commonly used resource for pathway analysis and contains data associated with pathways, genes, genomes, chemical compounds and reaction information. Part II of the report will focus on comparing the KEGG pathways for the entire RePOOPulate ecosystem, in search of keystone bacterial species and pathways, as well as species that may be biochemically redundant.

The third stage of the project focused on determining whether the bacterial genes included in RePOOPulate provide adequate coverage of the necessary biochemical pathways without high levels of genetic redundancy. Part III of the report shows the entire RePOOPulate community's coverage of the KEGG pathways as compared to that of a “healthy” human microbiome. This allowed for an examination of the overall coverage of the KEGG pathways to determine how close the RePOOPulate community emulates the true microbiota of the human gut.

Part I: Redundancy within Strain Pairs

### Methods

### Mauve Alignment

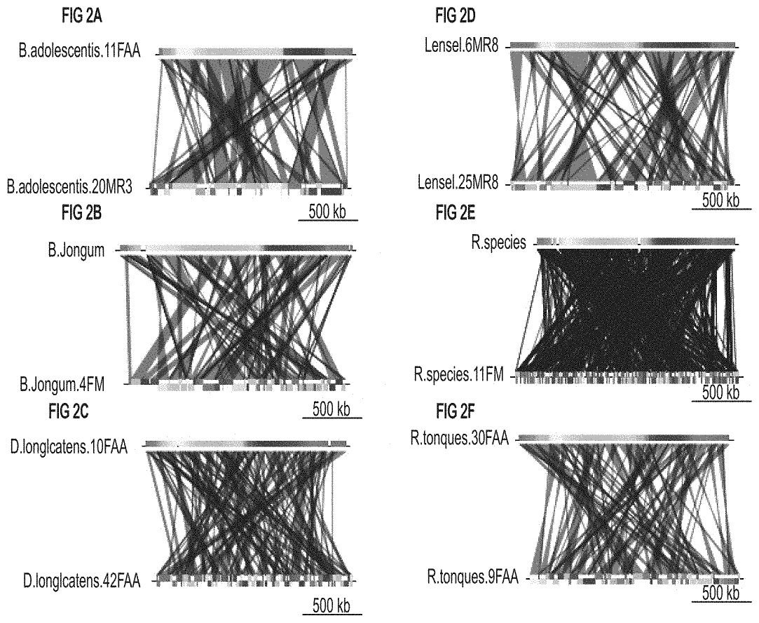

The original RePOOPulate prototype ecosystem included six species of bacteria with two separate strains, for a total of twelve bacterial strains. The whole genome data for both strains of these six species of bacteria were compared to test for redundancy. The pairs of genomes were aligned and compared using the progressive Mauve function of the genome alignment visualization tool Mauve. The resulting alignment backbone files were loaded into R and the package genoPlotR (pseudo-code provided) was used to create more dynamic images than those provided by Mauve (FIG. 2). Following alignment, strains for each species were assigned as either strain A or strain B to simplify further analysis of comparison results (Table 1).

FIG. 2 shows sequence alignment diagrams for mauve alignments, showing the alignment of the strain pairs for the six species analyzed in Part I and were created using Mauve and the R package genoPlotR. FIG. 2A shows Bifidobacterium adolescentis sequence comparison of strain A to strain B. FIG. 2B shows Bifidobacterium longum sequence comparison of strain A to strain B. FIG. 2C shows Dorea longlcatena sequence comparison of strain A to strain B. FIG. 2D shows Lactobacillus casei sequence comparison of strain A to strain B. FIG. 2E shows Ruminococcus torques sequence comparison of strain A to strain B. FIG. 2F shows Ruminococcus obeum sequence comparison of strain A to strain B.

Table 1 shows strain designation for part I, specifically determining redundancy within strain pairs. Identification of the strains referred to as strain A and strain B for each of the pairwise comparisons of the six species for which two strains were included in the original RePOOPulate ecosystem. Names in the table indicate the name given on the RAST server and bracketed numbers indicate the RAST genome ID number.

TABLE 1

| | Bifidobacterium | Bifidobacterium | [symbol] | Lactobacillus | Ruminococcus | [symbol] |
|---|---|---|---|---|---|---|
| Strain | [symbol] | longum | longicatena | casei | obeum* | torques |
| A | Bifidobacterium | Bifidobacterium | [symbol] | Lactobacillus | [symbol] | [symbol] |
| | [symbol] | [symbol] | [symbol] | [symbol] | species | torques 30FAA |
| | 11FAA | (6666666.437) | (6666666.43741) | (6666666.43739) | (6666666.437) | (6666666.43778) |
| | (6666666.43742) | | | | | |
| B | Bifidobacterium | Bifidobacterium | [symbol] | Lactobacillus | [symbol] | [symbol] |
| | [symbol] | [symbol] | [symbol] | [symbol] | sp. [symbol] | torques [symbol] |
| | [symbol] | (6666666.43669) | [symbol] | (6666666.43773) | (6666666.43778) | (6666666.43740) |
| | (6666666.43755) | | (6666666.43792) | | | |

### Comparison Using SEED Viewer

The draft genomes used in this analysis had been previously annotated and stored on the RAST server. RAST uses subsystem-based annotation, which identifies protein-encoding, rRNA and tRNA genes, assigns functions to the genes, predicts which subsystems are represented in the genome and uses this information to reconstruct the metabolic network. A subsystem is defined as a collection of functional roles, which together implement a specific biological process or structural complex. The subsystems-based approach is built upon the principle that the key to improved accuracy in high-throughput annotation technology is to have experts annotate single subsystems over the complete collection of genomes, rather than having an annotation expert attempt to annotate all of the genes in a single genome. The annotated genomes are maintained in the SEED environment, which supports comparative analysis. Following genome pair alignment and visualization, functional and sequence comparison of each strain pair was completed using the SEED Viewer accessed through the RAST server.

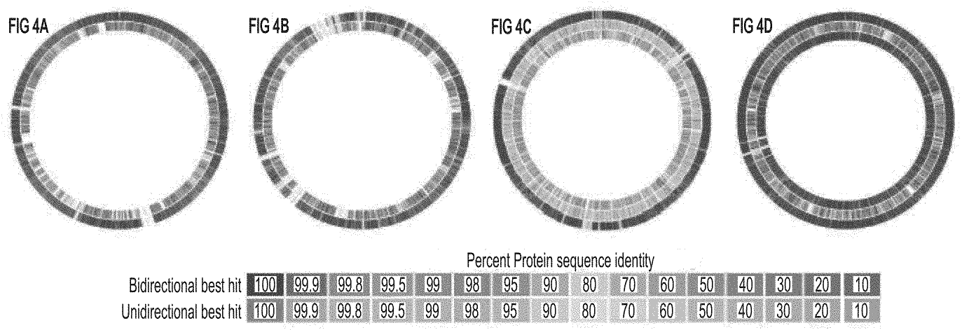

Functional comparison was used to identify subsystem-based differences using the annotated draft sequences. The functional comparison output provided consists of a table of identified subsystems indicating which subsystems were shared and which were unique to only one strain. The results of each of the six comparisons were exported in tab-separated value tables and examined in Microsoft Excel. A sequence comparison was then completed using the SEED Viewer to examine protein sequence identity and determine average genetic similarity. The image outputs were downloaded in graphics interchange format (gif) and textual results of this comparison were exported as tab-separated value tables and examined in Microsoft Excel. Protein sequence identity was examined both with and without the inclusion of hypothetical protein data. Sequence comparison was completed using both strain A as a reference and strain B as a reference since results differed slightly when different strains were used. When possible, strains were also compared to nearest available taxonomic neighbor in order to compare protein sequence similarity to that found in other bacterial strains within the same genus or species (FIG. 4). Data suggested that the genome size and the number of contigs could be confounding factors in the results for sequence comparison. This was examined using linear modeling in R. The data in Table 6 was saved as a comma-separated value file and loaded into R. Two linear models were fitted to compare the average percent protein sequence identity to genome size and to number of contigs (pseudo-code provided).

FIG. 4 shows SEED viewer sequence comparison figures for the closest available species match. FIG. 4A shows a comparison of reference Bifidobacterium adolescentis strain A to strain B (outer ring) and Bifidobacterium adolescentis (1680.3) (inner circle). FIG. 4B shows the sequence comparison of Bifidobacterium longum strain A to strain B (outer ring) and Bifidobacterium longum DjO10A (inner ring). FIG. 4C shows the sequence comparison of Dorea longicatena strain A to strain B (outer ring) and Dorea formicigenerans ATCC27755 (middle ring) and Dorea longicatena DSM 13814 (inner ring). FIG. 4D shows sequence comparison of Lactobacillus casei strain B to Lactobacillus casei strain A (outer ring) and Lactobacillus casei ATCC 334 (middle ring) and Lactobacillus casei BL23 (inner ring). No Ruminococcus species were openly available for comparison purposes on the SEED viewer.

Table 6 shows summary statistics for strains analyzed in Part I, showing redundancy within strain pairs. Table 6 includes the size of the genome in number of base pairs, the number of contigs in the draft sequences used, the percent similarity to the closest match based on full-length 16S sequence alignment (inferred from original RePOOPulate paper), the total number of subsystems, coding sequences and RNAs identified using the SEED viewer, and the average percent protein sequence identity calculated in Microsoft Excel using data obtained from the Seed viewer (the listed strain is the reference strain for the comparison of strain pairs).

TABLE 6

| | | | % Identity | | Number of | | Average % |
|---|---|---|---|---|---|---|---|
| | Genome | Number of | to closest | Number of | Coding | Number of | Protein Sequence |
| Strain | Size | Contigs | 16s match | Subsystems | Sequences | RNAs | Identity |
| B. adolescentis A | 2297655 | 31 | 99.79% | 271 | 1986 | 64 | 95.71% |
| B. adolescentis B | 2241437 | 20 | 99.79% | 270 | 1905 | 66 | 98.63% |
| B. longum A | 2607832 | 51 | 99.86% | 285 | 2329 | 108 | 90.13% |
| B. longum B | 2437247 | 48 | 99.16% | 282 | 2152 | 82 | 95.52% |
| D. longicatena A | 2861439 | 93 | 99.62% | 279 | 2716 | 67 | 94.47% |
| D. longicatena B | 2810362 | 73 | 99.60% | 288 | 2686 | 61 | 94.38% |
| R. obeum A | 3788856 | 61 | 94.89% | 311 | 3615 | 75 | 49.07% |
| R. obeum B | 4266695 | 181 | 94.69% | 313 | 4025 | 58 | 46.03% |
| R. torques A | 3372026 | 53 | 99.15% | 303 | 3209 | 68 | 99.26% |
| R. torques B | 3366080 | 63 | 99.29% | 303 | 3206 | 68 | 99.12% |
| L. casei A | 3038156 | 48 | 99.47% | 352 | 3096 | 48 | 98.94% |
| L. casei B | 3047335 | 51 | 99.74% | 352 | 3102 | 52 | 98.82% |

### KEGG Pathway Analysis

KAAS (KEGG Automatic Annotation Server) was used to provide functional annotation of the genes in the draft genomes (contigs) by BLAST comparison against a manually curated set of ortholog groups in the KEGG GENES database. The amino acid FASTA files for the twelve genomes examined in Part I were uploaded to KAAS and annotated using the prokaryotes gene data set and the bi-directional best hit assignment method, recommended for draft genome data. The result contains KEGG Orthology (KO) assignments and automatically generated KEGG pathways. The lists of KO assignments (KO IDs) were downloaded and compared in Microsoft Excel. Lists of KO IDs shared between pairs of strains and lists of KO IDs specific to one strain but not the other were created using Microsoft Excel spreadsheet tables. These lists were then used to create a final list of KO IDs with weights that matched the number of replicates of a KEGG orthology assignment and colors determined by whether or not an ID was shared (green for shared, red for strain A, blue for strain B). The final lists (one for each of the six species) were then imported into the program iPath2.0: interactive pathway explorer. iPath is a web-based tool for the visualization, analysis and customization of the various pathways maps. The current version provides three different global overview maps including: a map of metabolic pathways, constructed using 146 KEGG pathways, giving an overview of the complete metabolism in biological systems; a regulatory pathways map, which includes 22 KEGG regulatory pathways; and a biosynthesis of secondary metabolites map, which contains 58 KEGG pathways.

The lists of KO IDs created were matched to the internal list used by iPath2.0 before mapping; this removed several KO IDs since iPath2.0 does not include all available KO IDs in the mapping program. The matched lists were then used to create custom maps for each of the six strain comparisons. Lists of conflicts, in which KO IDs with different colors or weights fell within the same pathway, were automatically created through the mapping process for each strain comparison. The ipath2.0 program automatically resolves these conflicts by random choice. This method of resolution was not ideal for this study design; instead conflicts were resolved manually. Any color conflicts were resolved to be green, since a conflict in color meant the pathway was shared and therefore not unique. Any conflicts between weights were resolved by taking the average weight (rounded to the nearest whole number) or the least conflicting weight, in cases where a single KO ID conflicted with multiple KO IDs of the same weight. The final maps and lists of unique KO IDs were then analyzed to determine which pathways were unique to one strain and whether redundancies could be removed.

### Results

### Mauve Alignment

Alignments provided a good visualization of the number of contigs and similarities between species strains. Based on visualization of the alignments, Bifidobacterium adolescentis strains and Lactobacillus casei strains appeared to be very similar. Alignment visualization also showed an early indication that the Ruminococcus obeum strains are more dissimilar than the other five species examined. Difference is alignment could reflect true strain differences, but could also be the result of incorrectly ordered contigs, which appear as genome rearrangements. Alignment figures can be found in FIG. 2.

### Functional Comparison Using SEED Viewer

Table 2 shows SEED viewer functional comparison results. A summary of the functional comparison of pairs of bacterial strains from six different bacterial species based on subsystem annotation; numbers indicate the number of subsystems roles identified to be present in strain A and not strain B, present in strain B and not strain A, or present in both strains and the total number of subsystems roles identified for each species comparison.

TABLE 2

| | Bifidobacterium | Bifidobacterium | [symbol] | Lactobacillus | Ruminococcus | [symbol] |
|---|---|---|---|---|---|---|
| Active in | adolescentis | [symbol] | [symbol] | casei | torques | [symbol] |
| A not B | 3 | 14 | 8 | 0 | 3 | 125 |
| B not A | 3 | 5 | 17 | 1 | 2 | 122 |
| A & B | 1184 | 1234 | 1235 | 1706 | 1420 | 1262 |
| Total | 1190 | 1253 | 1260 | 1707 | 1425 | 1509 |

Functional comparison of the strain pairs for the six bacterial species with two different strains revealed comparatively: very high functional redundancy in three species, high functional redundancy in two species and low functional redundancy in one species. The highest level of functional redundancy using a subsystem-based method of comparison was seen in the comparison of the Lactobacillus casei pairs. The only difference in functional subsystems was identified to be present in strain B and not strain A and involved lactose and galactose uptake (Table 3). The lowest level of redundancy was seen in the comparison of the Ruminococcus obeum strain pairs where 247 differences in functional subsystem roles were identified over a broad range of subsystems and categories. Comparison of both Ruminococcus torques and Bifidobacterium adolescentis strain pairs revealed only five and six differences between strains respectively, a comparatively very high level of redundancy (Table 3). The Bifidobacterium longum comparison of strain pairs showed slightly less redundancy with 19 differences in functional subsystem roles between strain A and strain B, 14 of which were present in Bifidobacterium longum strain A not B and only 5 of which were present in stain B not A. The comparison of Dorea longicatena strain pairs revealed 8 subsystem roles present in strain A not B and 17 subsystems present in strain B not A. A full list of differences in the comparison of functional subsystems for the Bifidobacterium longum and Dorea longicatena strain pairs is available in Table 8.

TABLE 8B

| Strain | Category | Subcategory | Subsystem | Role |
|---|---|---|---|---|
| A | Amino Acids and | Alanine, serine, | Glycine and Serine | L-serine dehydratase, |
| | Derivatives | and glycine | Utilization | alpha subunit (EC |
| | | | | 4.3.1.17) |
| | Carbohydrates | Di- and | Sucrose | PTS system, sucrose- |
| | | oligosaccharides | utilization | specific IIA component |
| | | | | (EC 2.7.1.69) |
| | Clustering-based | No subcategory | CBSS- | His repressor |
| | subsystems | | 393121.3.peg.1913 | |
| | Cofactors, | No subcategory | Thiamin | Substrate-specific |
| | Vitamins, | | biosynthesis | component YkoE of |
| | Prosthetic Groups, | | | thiamin-regulated ECF |
| | Pigments | | | transporter for |
| | | | | HydroxyMethylPyrimidine |
| | | | | Transmembrane |
| | | | | component YkoC of |
| | | | | energizing module of |
| | | | | thiamin-regulated ECF |
| | | | | transporter for |
| | | | | HydroxyMethylPyrimidine |
| | RNA Metabolism | RNA processing | 16S rRNA | Penicillin-binding protein 3 |
| | | and modification | modification within | |
| | | | P site of ribosome | |
| | | Transcription | Transcription | RNA polymerase sigma |
| | | | initiation, bacterial | factor RpoE |
| | | | sigma factors | |
| | Virulence, Disease | Resistance to | Arsenic resistance | Arsenical resistance |
| | and Defense | antibiotics and | | operon repressor |
| | | toxic compounds | | |
| B | Amino Acids and | Arginine; urea | Arginine and | Arginine decarboxylase |
| | Derivative | cycle, polyamines | Ornithine | (EC 4.1.1.19) |
| | | | Degradation | Ornithine decarboxylase |
| | | | | (EC 4.1.1.17) |
| | | Lysine, threonine, | Lysine degradation | Lysine decarboxylase (EC |
| | | methionine, and | | 4.1.1.18) |
| | | cysteine | | |
| | Carbohydrates | Di- and | Beta-Glucoside | PTS system, beta- |
| | | oligosaccharides | Metabolism | glucoside-specific IIA |
| | | | | component (EC 2.7.1.69) |
| | | | | PTS system, beta- |
| | | | | glucoside-specific IIB |
| | | | | component (EC 2.7.1.69) |
| | | | | PTS system, beta- |
| | | | | glucoside-specific IIC |
| | | | | component (EC 2.7.1.69) |
| | | One-carbon | Serine-glyoxylate | Fumarate hydratase class I, |
| | | Metabolism | cycle | aerobic (EC 4.2.1.2) |
| | Cell Wall and | Capsular and | Sialic Acid | Glucosamine-1-phosphate |
| | Capsule | extracellular | Metabolism | N-acetyltransferase (EC |
| | | polysaccharides | | 2.3.1.157) |
| | Clustering-based | No subcategory | RNA modification | GTPase and tRNA-U34 5- |
| | subsystems | | and chromosome | formylation enzyme TrmE |
| | | | partitioning cluster | |
| | Cofactors, | Biotin | Biotin biosynthesis | Biotin synthase (EC |
| | Vitamins, | | | 2.8.1.6) |
| | Prosthetic Groups, | | | |
| | Pigments | | | |
| | DNA Metabolism | DNA repair | DNA repair, | DNA-cytosine |
| | | | bacterial | methyltransferase (EC |
| | | | | 2.1.1.37) |
| | | DNA replication | DNA-replication | DNA polymerase III delta |
| | | | | prime subunit (EC 2.7.7.7) |
| | Phages, | Phages, Prophages | Phage capsid | Phage major capsid |
| | Prophages, | | proteins | protein |
| | Transposable | | Phage packaging | Phage portal protein |
| | elements, | | machinery | |
| | Plasmids | | Phage replication | DNA primase/helicase, |
| | | | | phage-associated |
| | | | Phage tail proteins | Phage tail length tape- |
| | | | | measure protein |
| | Stress Response | Heat shock | Heat shock dnaK | Signal peptidase-like |
| | | | gene cluster | protein |
| | | | extended | |

Table 8 shows a summary of SEED viewer functional comparisons. (A) shows Bifidobacterium longum. (B) Dorea longicatena. A summary of the subsystem based functional differences between strains A and B for Bifidobacterium longum and Dorea longicatena showing the category, subcategory, subsystem, and roles identified. The sections indicated on the row entitled ‘Phages, Prophages, Transposable Elements and Plasmids’ indicate differences related to phage elements.

Table 3 shows a summary of SEED viewer functional comparison. A summary of the subsystem based functional differences between strains A and B for Lactobacillus casei, Bifidobacterium adolescentis, and Ruminococcus torques showing the category, subcategory, subsystem and roles identified. Sections highlighted in grey indicate differences related to phage elements.

A key element to note is the large number of phage-related proteins and roles related to phages present in the comparisons (highlighted in grey text in Table 3 and Table 8). Phage related proteins were present in one strain but not the other for Bifidobacterium longum and Dorea longicatena and were present, but with different roles, in both strains of Bifidobacterium adolescentis and Ruminococcus obeum. These elements could help to explain the differences between these strain pairs. If one strain was infected with a phage while another remained unaffected, or strains were infected by different phages, this could cause the some of the differences in genes and functionality reported in this analysis. This is an excellent explanation of the strain divergence since phages are key horizontal gene transfer (HGT) mediators and an important pathway for gene introduction into the human gut microbiome.

Sequence Comparison using SEED viewer

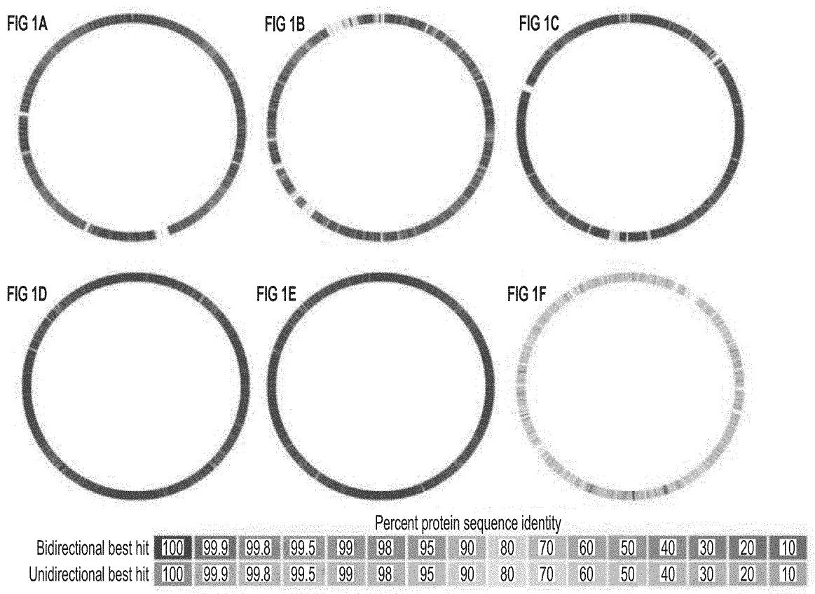

The sequence comparison for the strain pairs of the bacterial species for which two strains had been included in the original RePOOPulate ecosystem revealed similar results to the functional comparison. Five of the six species examined showed high to very high redundancy in their protein sequences. Comparison of the strain pairs for Bifidobacterium adolescentis, Bifidobacterium longum, Dorea longicatena, Lactobacillus casei and Ruminococcus torques all showed an average percent protein sequence identity of 95% or greater (see Table 7). The Ruminococcus obeum strain comparison by contrast had a much lower average percent protein sequence identity of between 45 and 62%, dependent upon whether or not hypothetical proteins were included in the comparison and which strain was used as the reference strain. The differences between the protein sequences can be clearly visualized in FIG. 1, which shows the percent protein sequence identity of strain B for each of the six species when strain A of the same species is used as a reference. The first five species are clearly in the 90% or greater range for the majority of the identified protein sequences, whereas the Ruminococcus obeum strains appear closer to the 50-60% range.

Table 7 shows a summary of SEED viewer sequence comparisons of pairs of bacterial strains from six different bacterial species based on percent protein sequence identity; numbers in brackets indicate comparisons with hypothetical proteins removed. Tables include the total number of proteins identified, the number of bi-directional and uni-directional hits, the total number of proteins with no hits (0%), the total number of proteins with perfect sequence match (100%), the number of proteins with high protein sequence identity (95%-99%), the number of proteins with low protein sequence identity (50% or less, not including those with no hits) and the average percent protein sequence identity. (A) summarizes the sequence comparisons with strain A as a reference strain. (B) summarizes the sequence comparisons with strain B as a reference strain.

FIGS. 1A and 1B show SEED viewer sequence comparison figures for strain pairs. Diagrams show comparison between strain A as a reference sequence and strain B. A) Bifidobacterium adolescentis sequence comparison of strain A to strain B. B) Bifidobacterium longum sequence comparison of strain A to strain B. C) Dorea longicatena sequence comparison of strain A to strain B. D) Lactobacillus casei sequence comparison of strain A to strain B. E) Ruminococcus torques sequence comparison of strain A to strain B. F) Ruminococcus obeum sequence comparison of strain A to strain B.

TABLE 7

| A | Sequence Comparison A to B | | | | | | | | | | | |
|---|---|---|---|---|---|---|---|---|---|---|---|---|
| Summary | B. | | B. | | D. | | R. | | R. | | L. | |
| Statistics | adolescentis | | longum | | longicatena | | obeum | | torques | | casei | |
| Total | 1986 | (1314) | 2329 | (1522) | 2716 | (1824) | 3615 | (2228) | 3209 | (1997) | 3096 | (2254) |
| Bi-directional | 1877 | (1288) | 2023 | (1424) | 2502 | (1725) | 2152 | (1625) | 3147 | (1978) | 3036 | (2225) |
| hits | | | | | | | | | | | | |
| Uni-directional | 35 | (17) | 126 | (66) | 110 | (74) | 535 | (378) | 41 | (15) | 30 | (26) |
| hits | | | | | | | | | | | | |
| Total w/0% | 74 | (9) | 180 | (32) | 104 | (25) | 928 | (225) | 21 | (4) | 30 | (3) |
| (no hit) | | | | | | | | | | | | |
| Total w/100% | 1595 | (1088) | 1504 | (1036) | 2413 | (1668) | 33 | (13) | 3096 | (1934) | 2944 | (2152) |
| 95-99% | 289 | (207) | 545 | (408) | 113 | (75) | 150 | (119) | 85 | (56) | 117 | (97) |
| 50% or less | 9 | (3) | 54 | (26) | 56 | (43) | 736 | (491) | 2 | (1) | 2 | (1) |
| Average % | 95.710 | (99.040) | 90.125 | (96.350) | 94.471 | (96.845) | 49.072 | (61.125) | 99.235 | (99.713) | 98.941 | (99.799) |
| protein id | | | | | | | | | | | | |

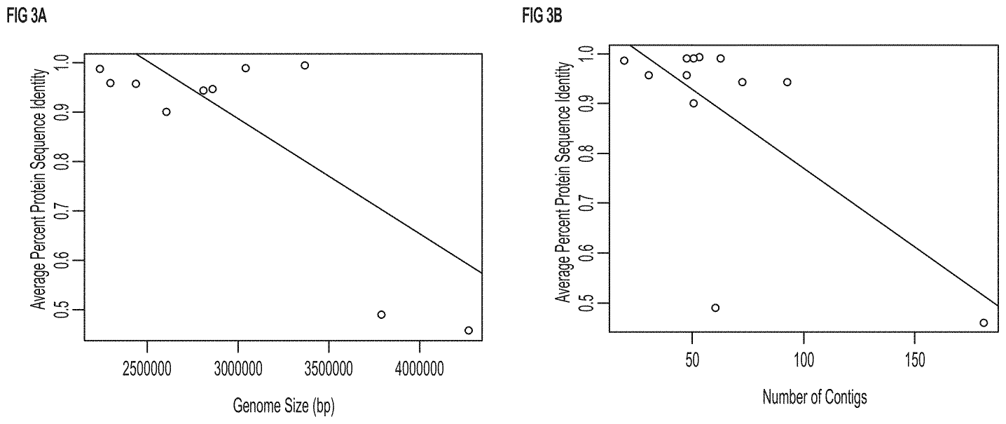

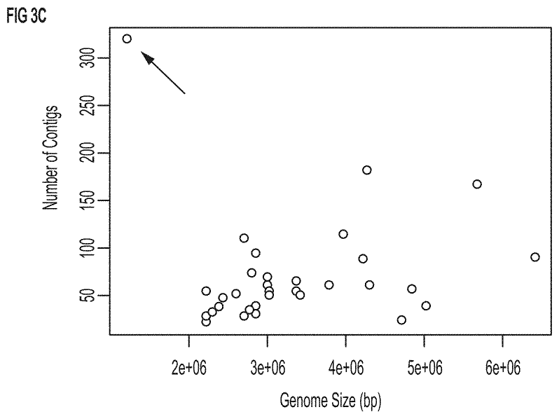

The linear models that were fitted for the comparison of the average percent protein identity to genomes size and number of contigs indicated that both of these factors could have confounded the results for the SEED sequence comparison to some level. The linear model for the comparison of genome size to average percent protein sequence identity had a p-value of 0.006 indicating a significant linear relationship. The linear relationship between the number of contigs and the average percent protein sequence identity was also significant with a p-value of 0.016. Scatterplots depicting these relationships can be found in FIG. 3.

FIG. 3 shows scatter plots for comparison using R. Plots were created in R using variations of the pseudo-code given below:

Pseudo-code for Linear Models

setwd(“/Users/folder/”)
Table<−read.table(file=”table.csv”, sep=”,”,header=TRUE)
LM1 <−lm(PercentProteinID~GenomeSize, data=Table)
summary(LM1)
plot(Table$GenomeSize,Table$PercentProteinID)
abline(LM1)

FIG. 3A shows a scatter plot of Genome Size versus Average Percent Protein Sequence Identity for the 12 bacterial genomes analyzed in Part I, with line showing the linear correlation between the two. Linear model has a p-value of 0.006144. FIG. 3B shows a scatter plot for the Number of Contigs versus Average Percent Protein Sequence Identity for the 12 bacterial genomes analyzed in Part I, with line showing the linear correlation between the two. Linear model has a p-value of 0.01629. FIG. 3C shows a scatter plot for Genome Size versus Number of Contigs for all 33 bacterial genomes. An outlier is Eubacterium rectale 18FAA, which appears to have had an error in sequencing.

KEGG Pathway Analysis

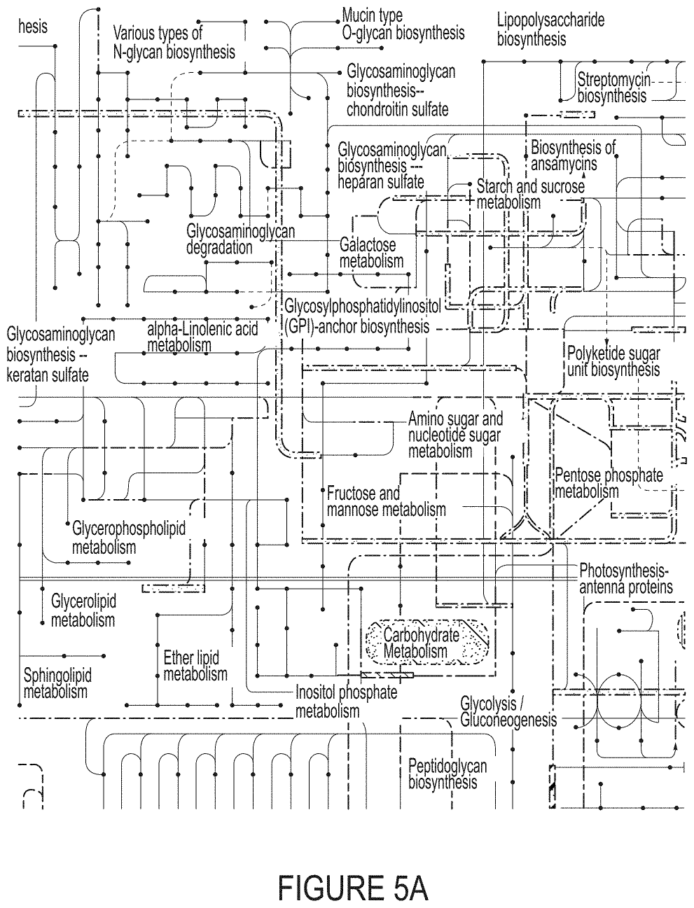

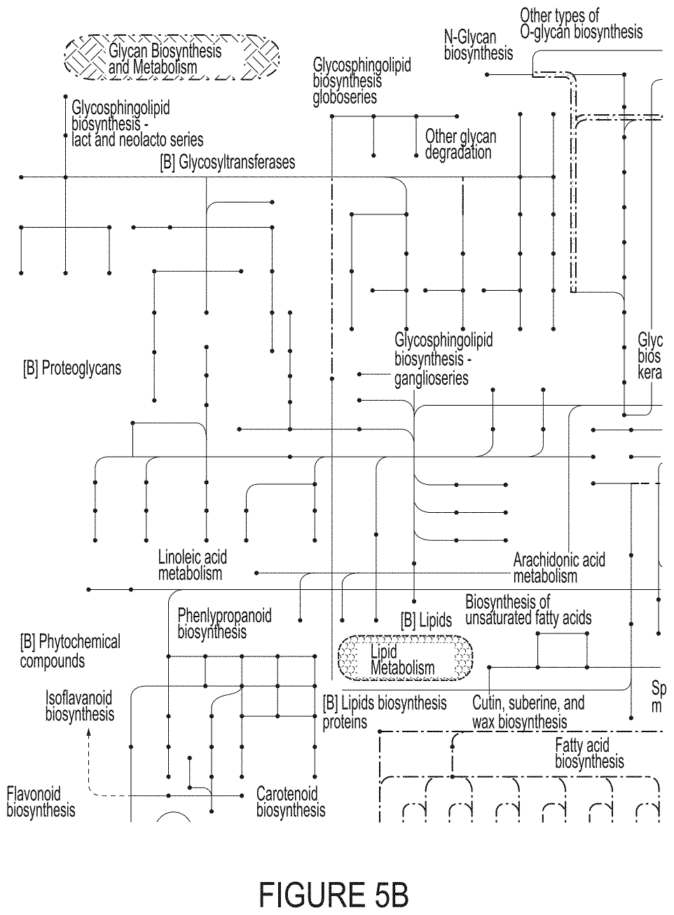

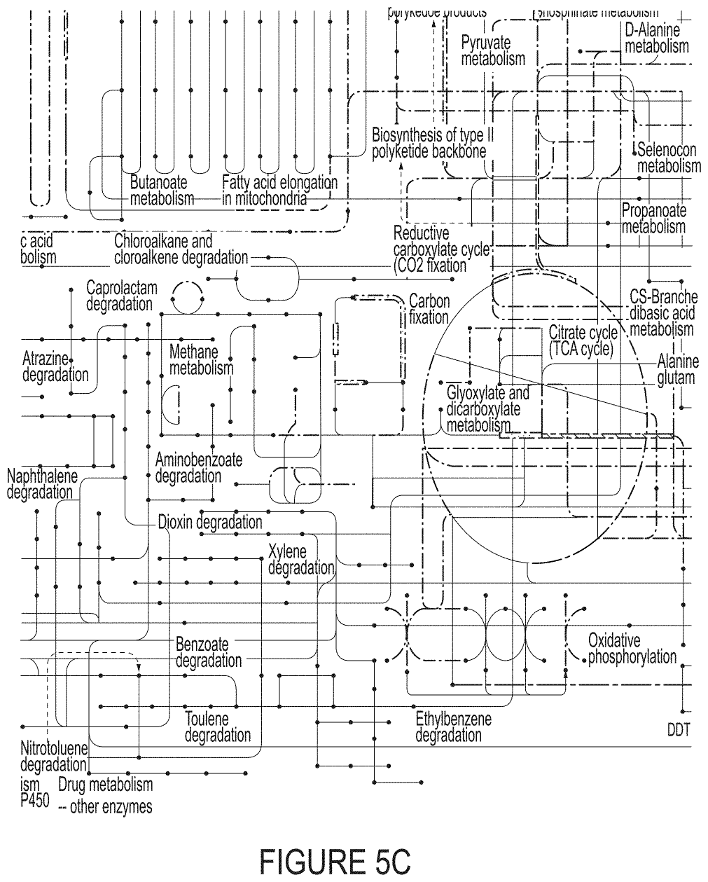

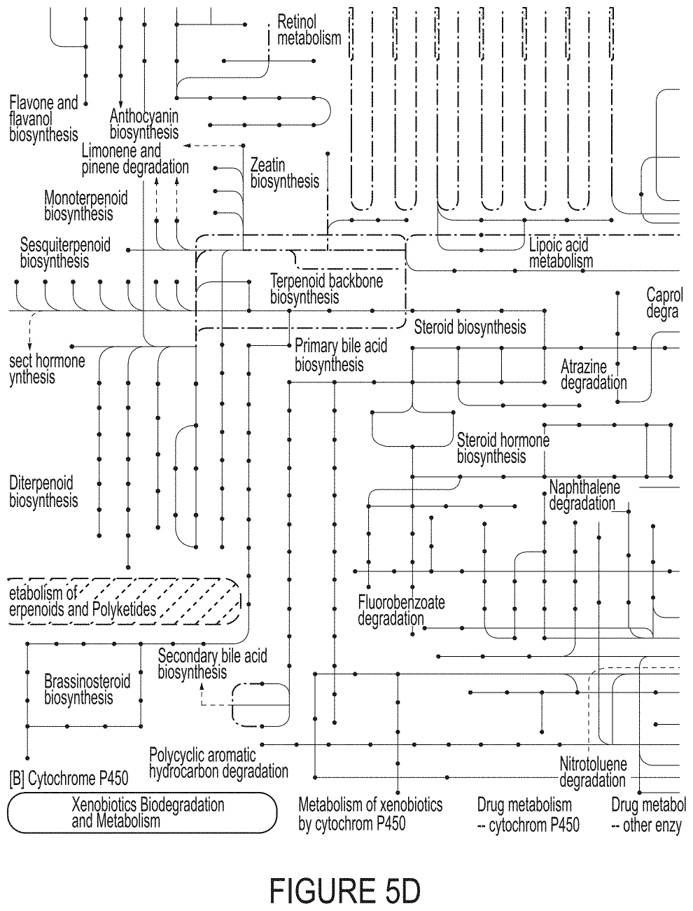

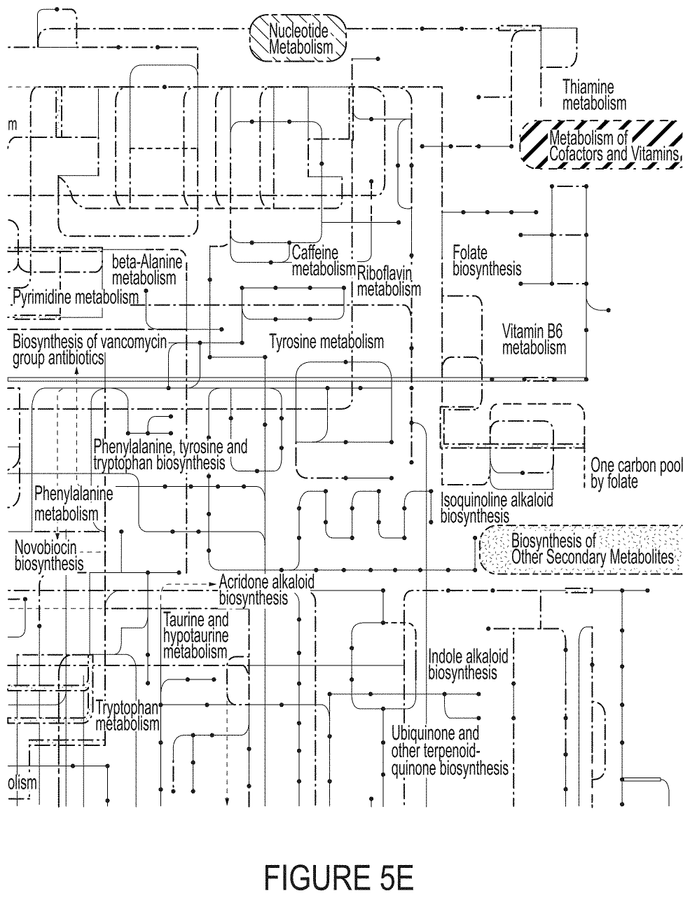

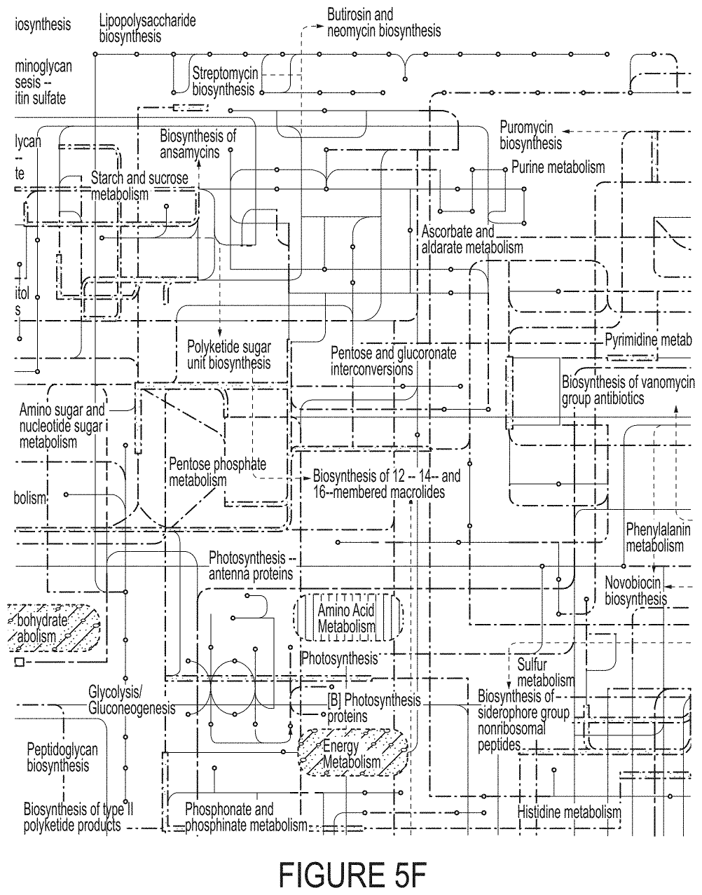

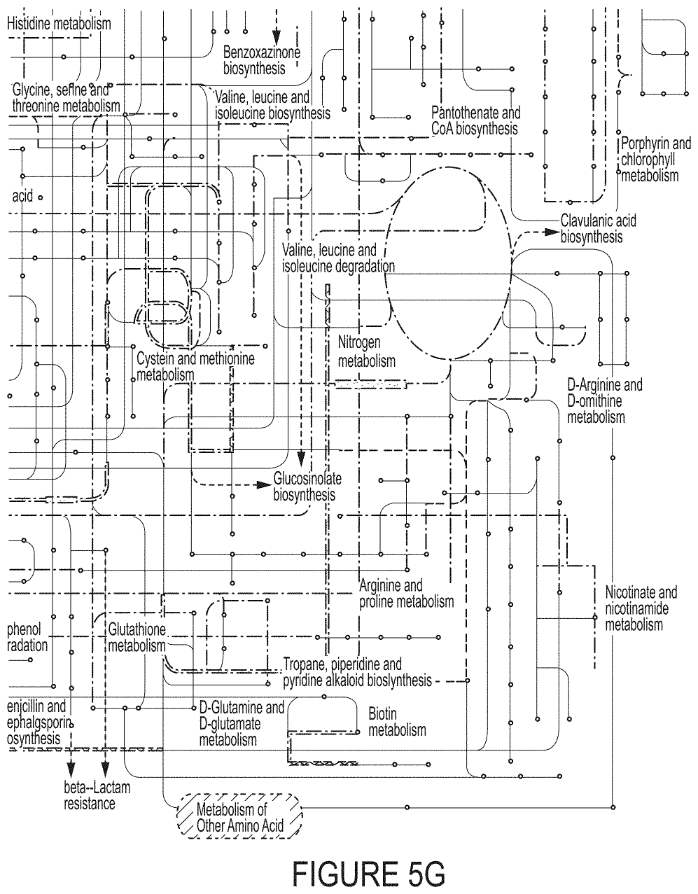

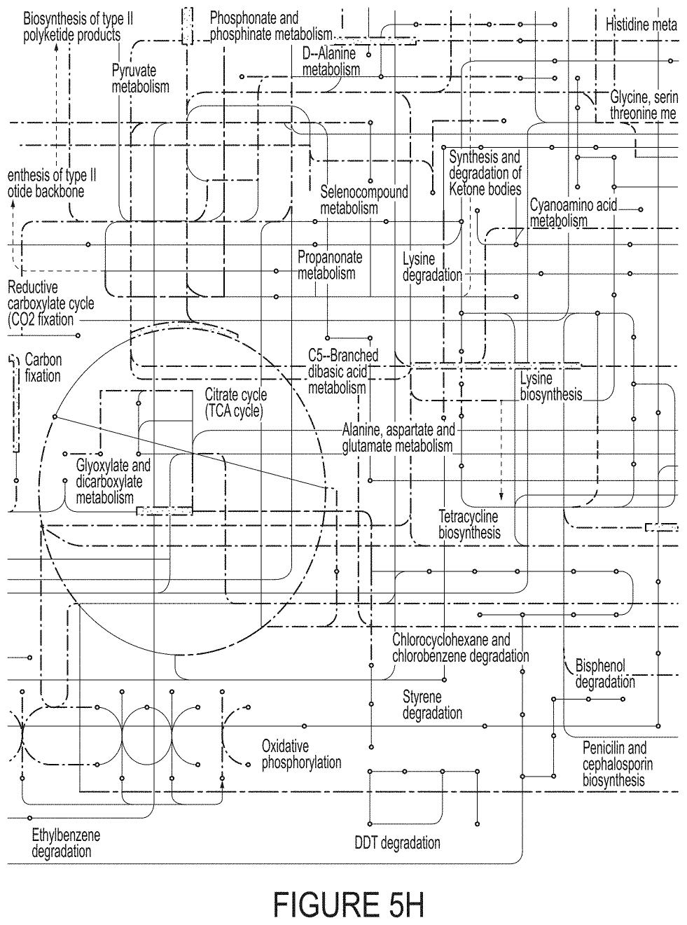

The KEGG pathway results confirmed the results of the functional and sequence comparisons using the SEED viewer. Comparison of KEGG Orthology for Bifidobacterium adolescentis, after ID matching to the internal iPath2.0 list and conflict resolution, revealed only three key differences in pathways that were present in strain B and not present in strain A. The Bifidobacterium longum KEGG comparison initially revealed 40 differences in KO IDS between strain A and B, however after matching and conflict resolution 5 KO IDs unique to strain A and 3 KO IDs unique to strain B, as well as 4 KO IDs with a higher number of replicates in strain A and 2 KO IDs with a higher number of replicates in strain B were found. The Lactobacillus casei KEGG pathway comparison revealed only one difference, a KO ID that was unique to strain B. This is consistent with the high level of redundancy between the Lactobacillus casei strains seen throughout this study. The Dorea longicatena comparison revealed 2 unique KO IDs for strain A and 6 unique KO IDs for strain B. The Ruminococcus torques KEGG comparison found only 2 unique KO IDs for each strain. A full list of the differences in KEGG Orthology assignments for these five species, and the pathway elements that they map to can be found in Table 9. The comparison of Ruminococcus obeum strains based on KEGG Pathway analysis revealed much the same results as the previous sections. The comparison found 43 unique IDs for strain A and 32 unique IDs for strain B, as well as 5 IDs with greater replication in strain A and 3 IDs with greater replication in strain B (FIG. 5). This is consistent with the low levels of redundancy seen in the SEED viewer comparison, indicating the necessity of both Ruminococcus obeum strains. These results, when combined with the results from the SEED viewer comparisons, indicate that strain A for Bifidobacterium adolescentis, Lactobacillus casei, and Dorea longicatena, as well as strain B for Bifidobacterium longum and Ruminococcus torques appear to be functionally redundant and could be removed from the ecosystem without causing an ecological imbalance.

FIGS. 5A-B shows KEGG pathway maps for comparing Ruminococcus obeum. FIG. 5A shows the metabolic pathway map. FIG. 5B shows the regulatory pathway map. KEGG pathway maps were generated using ipath2.0 for the comparison of Ruminococcus obeum strain A to strain B. Green lines represent shared pathways, red lines represent pathways unique to strain A or with greater repetition in strain A, blue lines represent pathways unique to strain B or with greater prepetition in strain B. Line weights are determined by number of repeats of KO IDs.

Table 9 shows a summary of the differences in KEGG pathways for five of the species compared in Part I. Table 9 includes the KO ID, the map(s) name (including biosynthesis of secondary metabolites, Sec. Biosynth.) and the specific pathway elements that are unique to one strain. Sections in blue indicate KO IDs and elements that are not unique to one strain but have a higher number of replicates in the strain indicated.

Part II: Redundancy within the RePOOPulate Ecosystem

Methods

Redundancy within the RePOOPulate ecosystem was examined in much the same way as the KEGG pathway comparison described above, but on a larger scale. KAAS (KEGG Automatic Annotation Server) was used to provide functional annotation of the genes in the draft genomes not included in Part I (21 further genomes). The lists of KO assignments (KO IDs) for each genome were downloaded and compared in a table in Microsoft Excel. A list of KO IDs found for all thirty-three species within the original RePOOPulate ecosystem, as well as a list of counts of the number of times a KO ID was found within the entire ecosystem was created from the Microsoft Excel table. These lists were then used to create a final list of KEGG IDs with weights that matched the number of replicates of a KEGG orthology assignment (KO ID). The list of KO IDs was then imported into the program iPath2.0: interactive pathway explorer and matched to the internal list used for by iPath2.0 before mapping; this removed several KO IDs from the list. This final matched list for all thirty-three species was used in Part III.

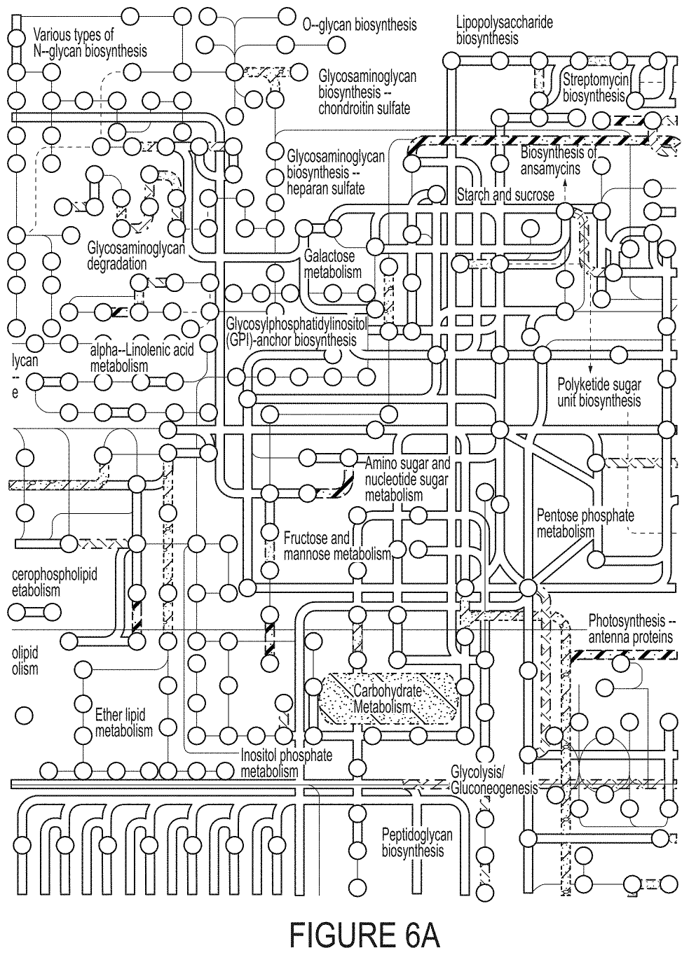

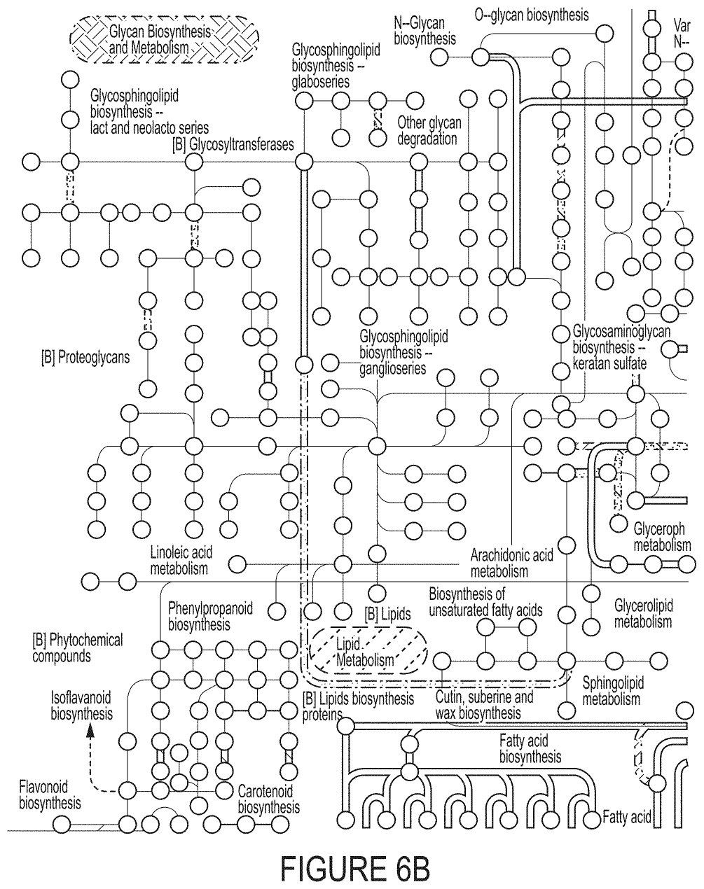

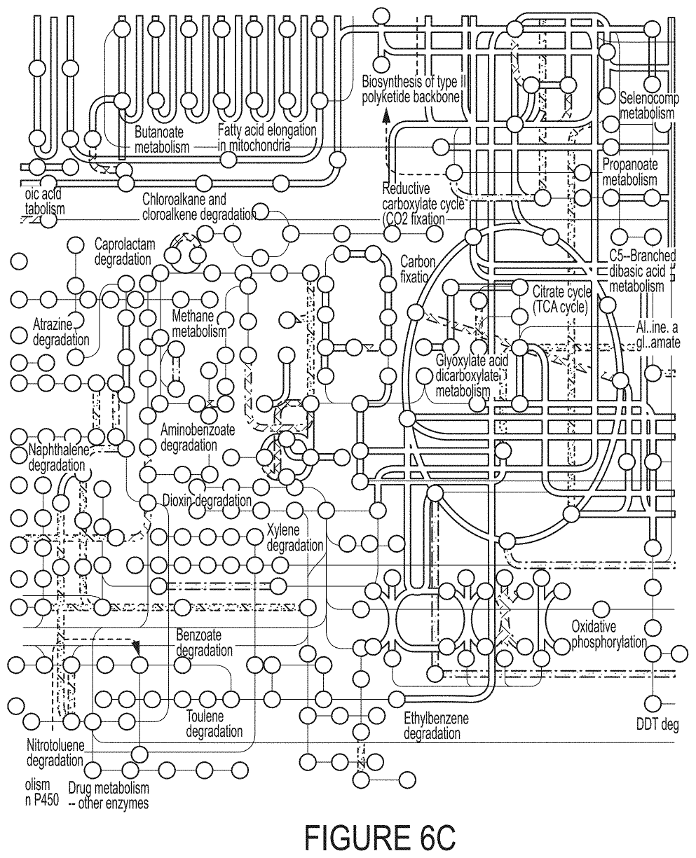

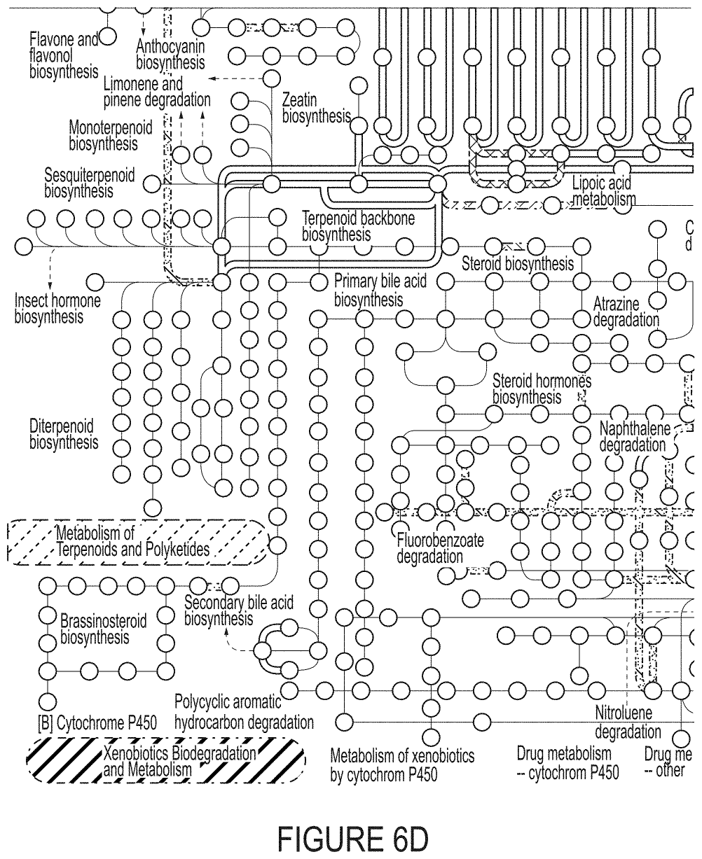

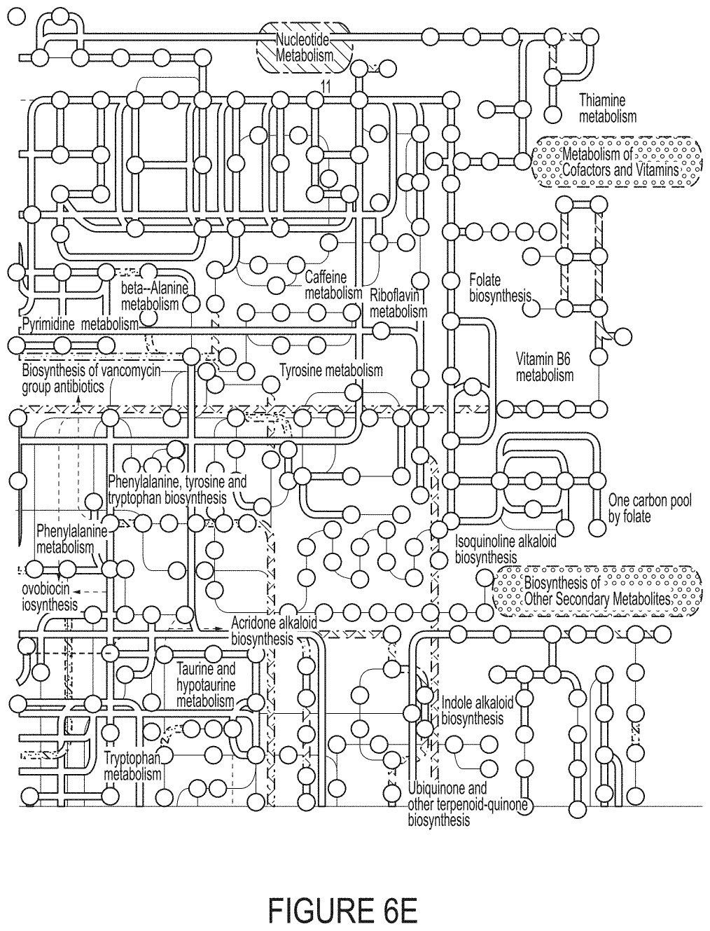

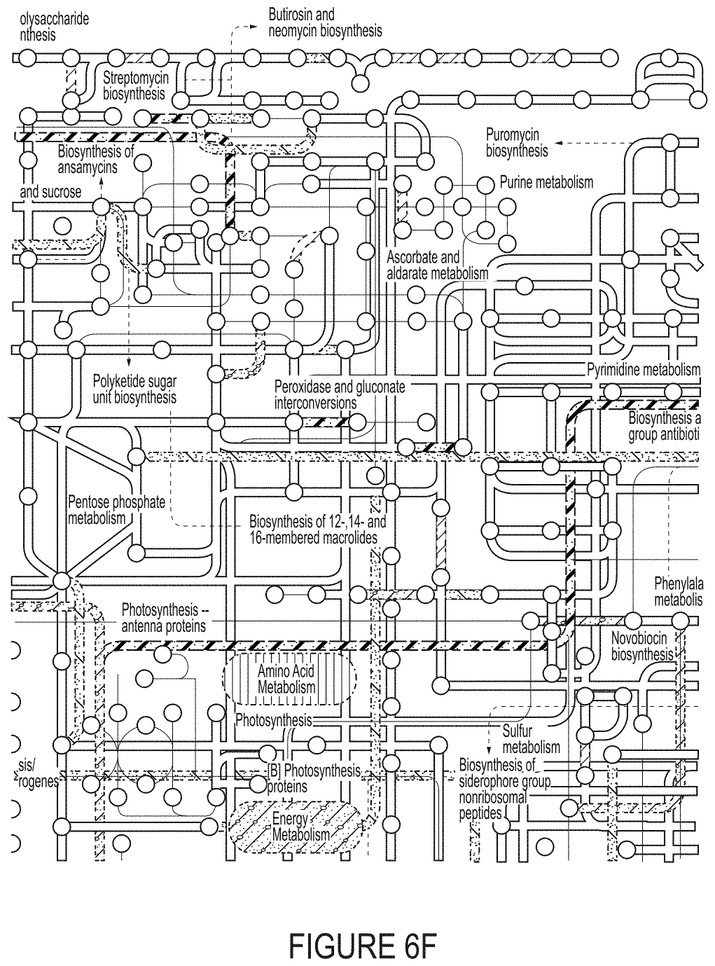

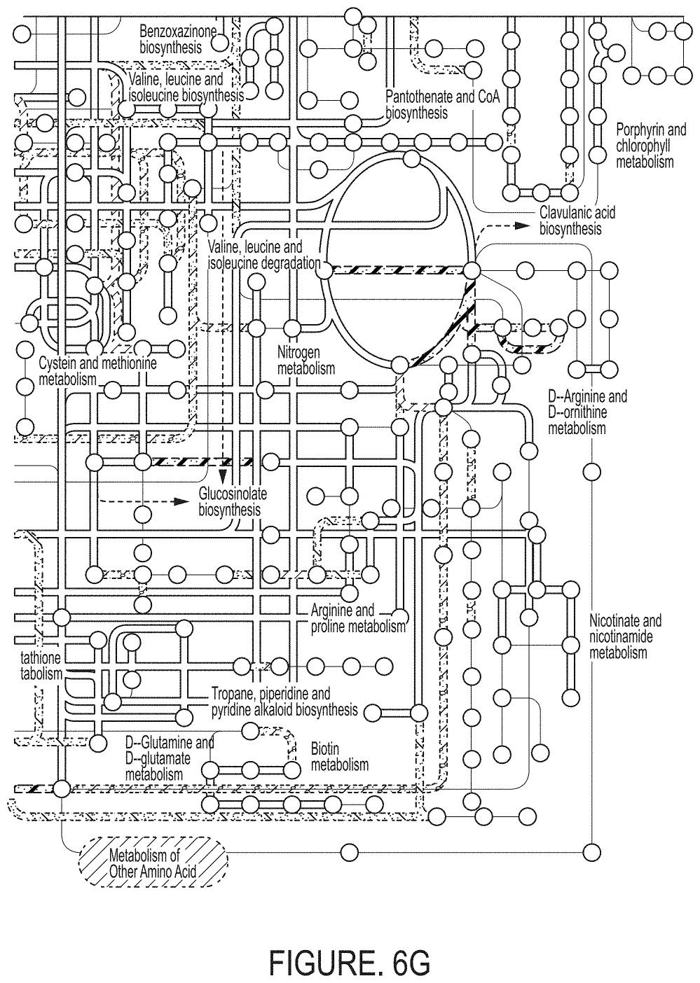

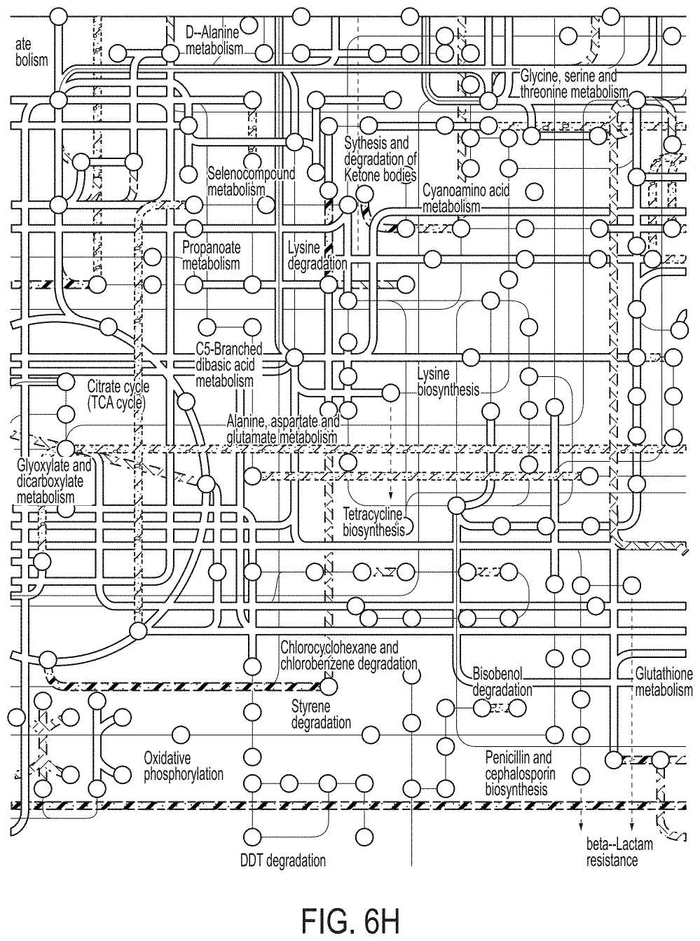

An updated list was next created following the removal of the eight species strains found to be redundant in Part I of this study (Table 4). The second list included only twenty-five different bacteria. A list of matched KO IDs for this smaller ecosystem was created, as well as lists of KO IDs specific to a single species, shared by two species, shared by three species, shared by four species and shared by five or more species. A list of counts of the number of replicates for each KO ID was also created. The lists of KO IDs shared by 1, 2, 3, 4, and 5 or more species were each color coded (purple, blue, green, red and black respectively) and imported into iPath2.0. Conflicts between colors were resolved as the color of the highest number of species it conflicted with, i.e., if a pathway had a conflict between red (4 species) and blue (2 species) it would resolved as red. The final metabolic pathway map was examined (FIG. 6) and counts of the number of nodes shared between each color were counted. Nodes in the map correspond to variout,32s chemical compounds and edges represent series of enzymatic reactions or protein complexes. Maps were also created for 1, 2, 3 and 4 species individually to obtain the number of pathway elements (edges) that their KO IDs mapped to (Table 10).

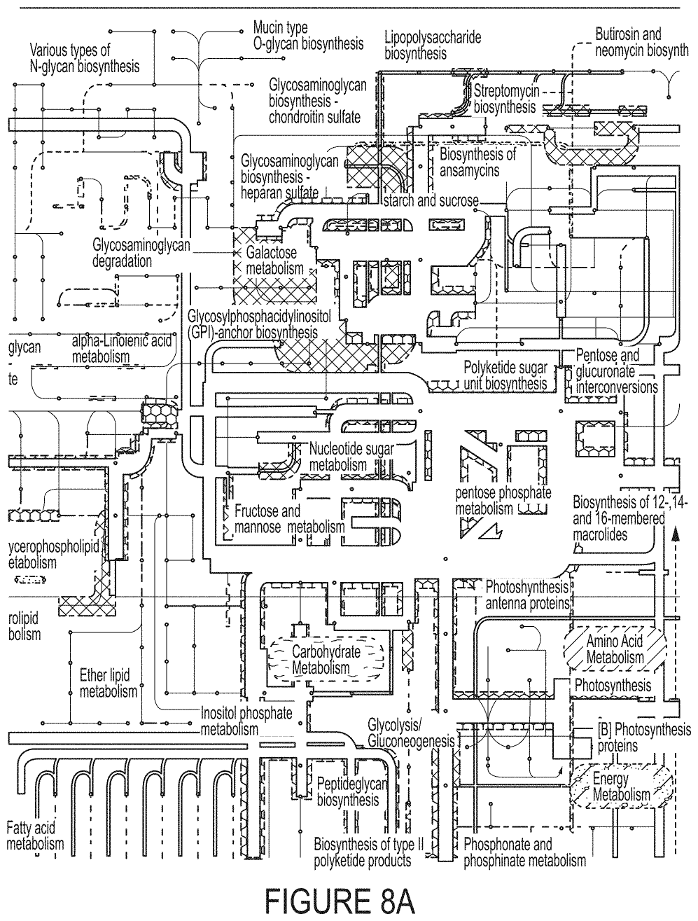

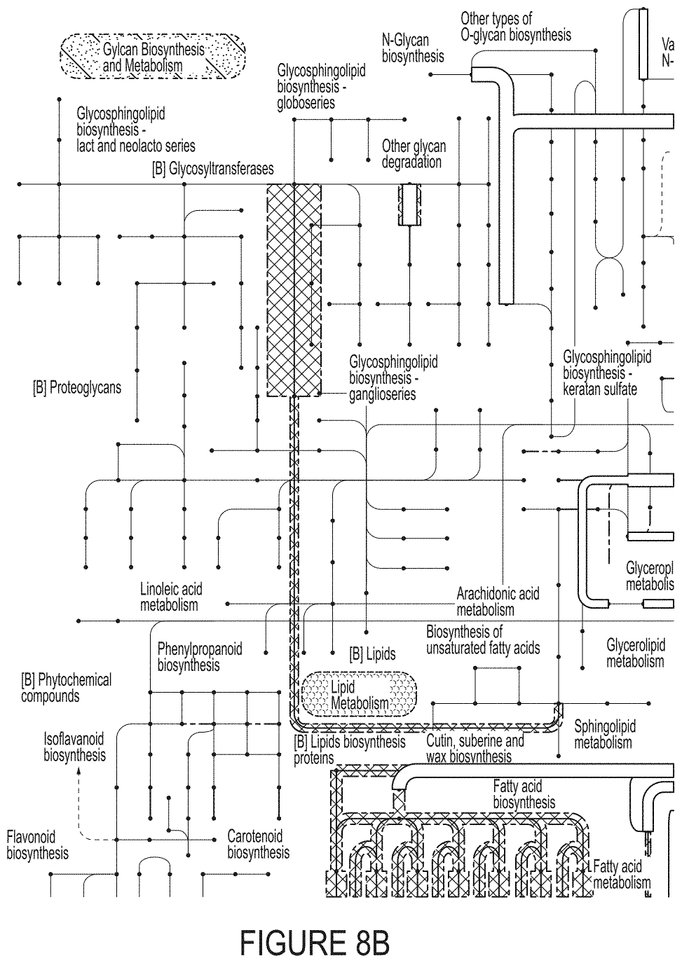

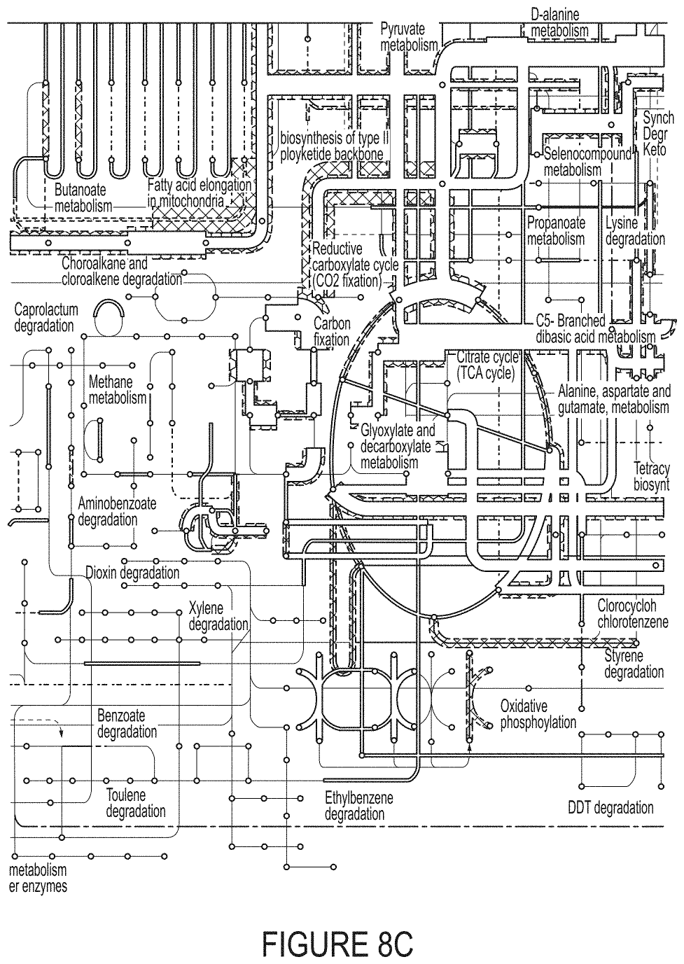

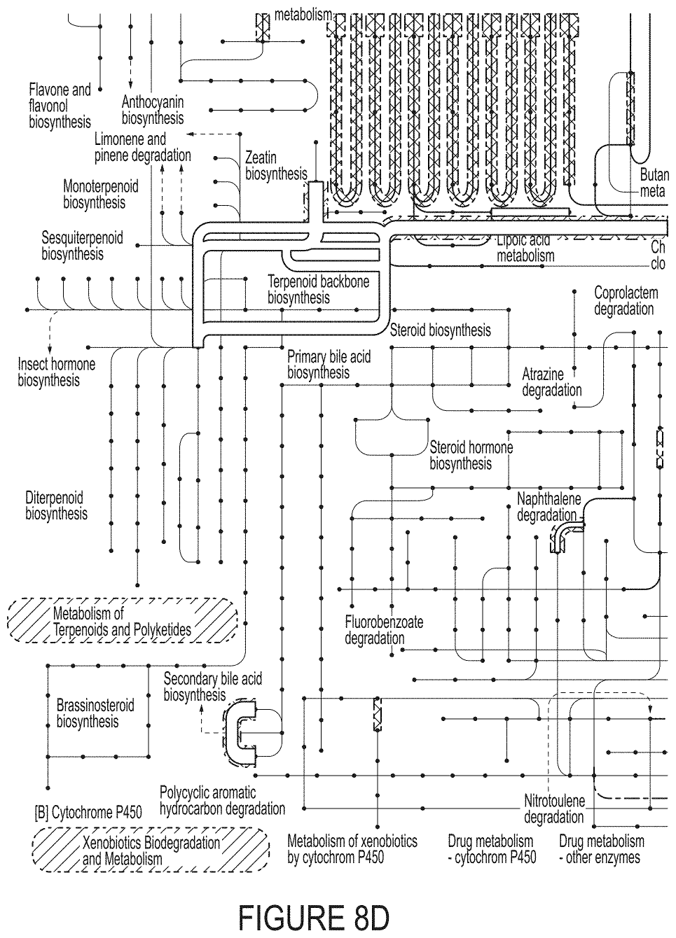

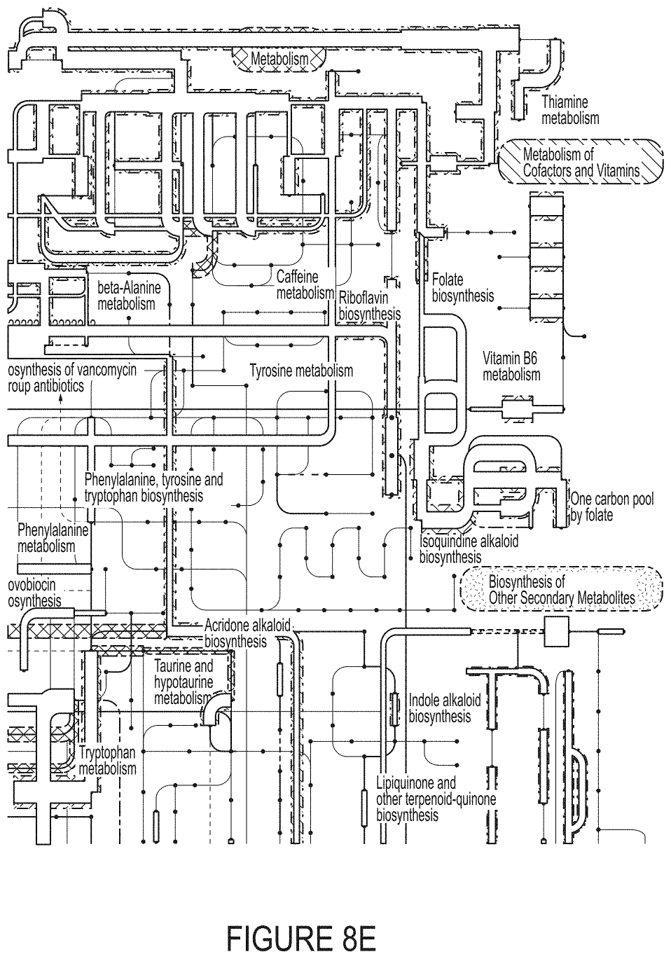

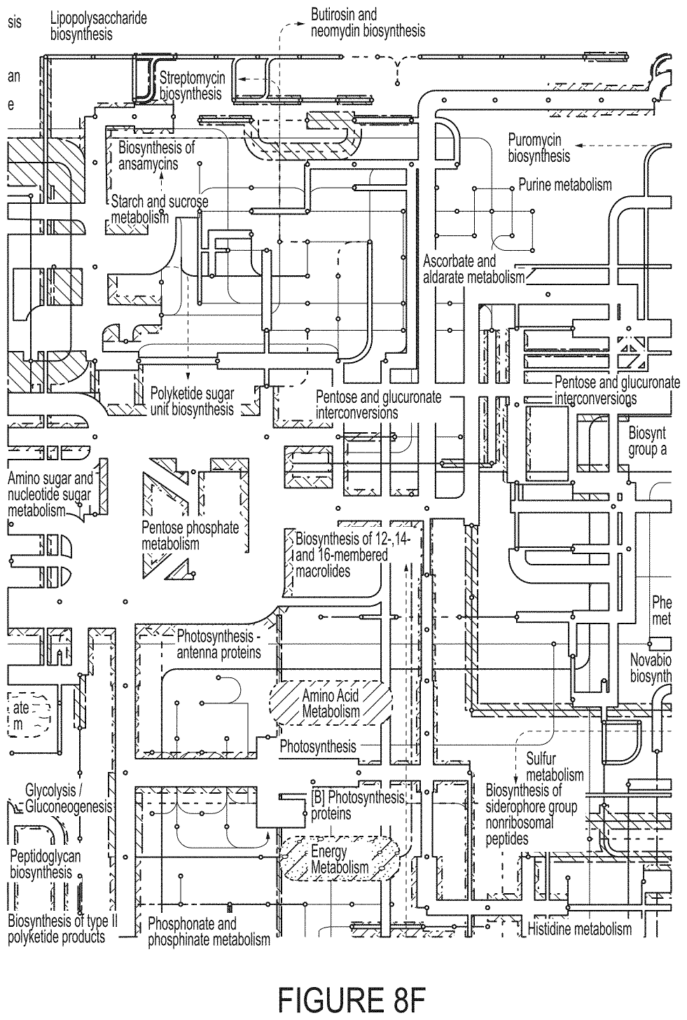

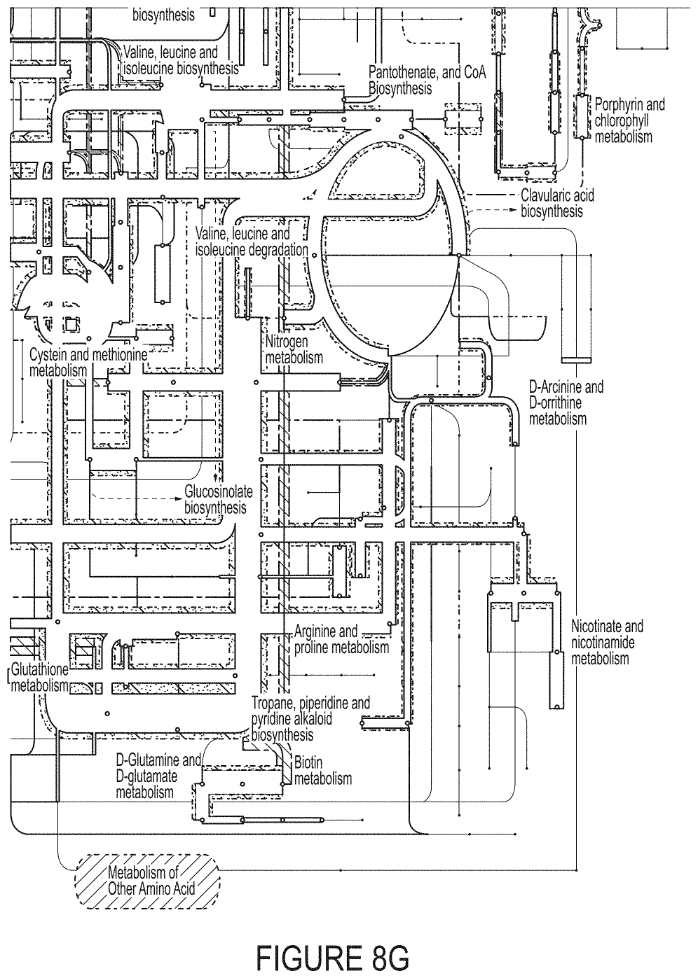

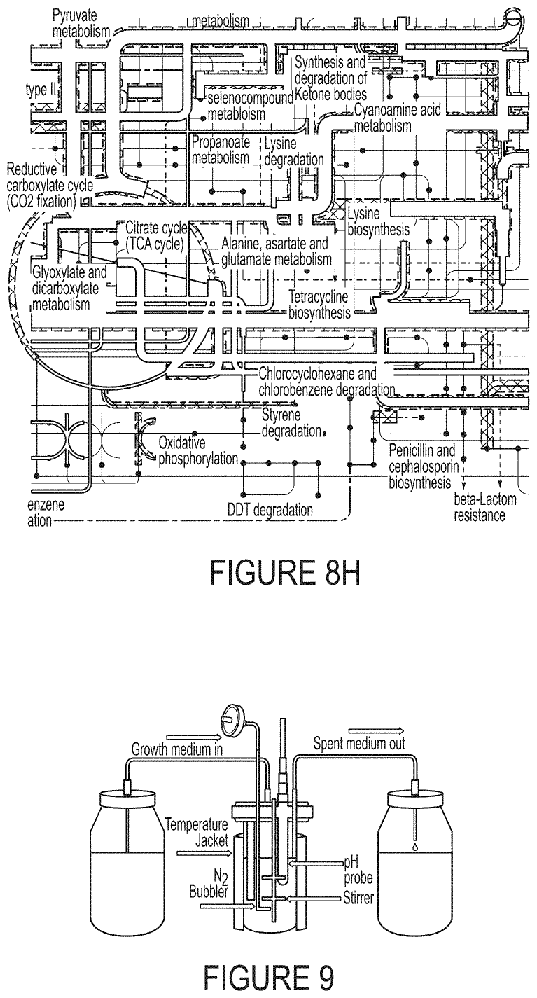

Table 10 shows element counts for ipath2.0 KEGG comparison pathways shared by one, two, three or four species. A summary of the results for the comparison of the RePOOPulate species after redundant strains for Part A were removed (includes 25 species), looking at the pathways shared by one, two, three and four species. Includes the number of pathway elements selected on each of the tree maps, and the counts for the number of unique nodes and shared nodes for the metabolic map (FIG. 8). Unique nodes were counted if the nodes were only part of a pathway that include the number of species shown, nodes shared by greater than four (>4) species were counted if one or more colored lines and a black line shared a node, nodes shared by 1/2/3/4 species were counted where two different colored lines shared a node, i.e. blue (two species) and green (three species).

FIG. 6 shows the metabolic pathway map for ipath 2.0 KEGG comparison of pathways shared by one, two, three or four species. Full metabolic pathway map for the comparison of the RePOOPulate species after redundant strains for Part I were removed (includes 25 species), showing metabolic pathways shared by one, two, three, or four species. Purple lines correspond to unique pathways shared by a single species, blue lines correspond to metabolic pathways shared by two species, green lines correspond to pathways shared by three species, red lines correspond to pathways shared by four species and black lines are all other pathways within the system (>4 species). Line weights were chosen for ease of visualization and do not reflect the number of copies of the KEGG orthology IDs.

TABLE 10

| | Pathways | | | | Nodes | | | |
|---|---|---|---|---|---|---|---|---|
| | | | Biosynthesis | | | Shared | Shared | |
| Number | | | of Secondary | | Unique | by >4 | by 1/2/3/4 | |
| of Species | Metabolic | Regulatory | Metabolites | Total | Nodes | species | species | Total |
| 1 | 98 | 58 | 24 | 180 | 96 | 46 | 11 | 153 |
| 2 | 80 | 111 | 27 | 218 | 44 | 55 | 23 | 122 |
| 3 | 40 | 55 | 6 | 101 | 20 | 26 | 10 | 56 |
| 4 | 54 | 48 | 12 | 114 | 24 | 48 | 10 | 82 |

The list of KO IDs specific to a single species revealed that only twenty-two of the twenty-five included bacteria had unique KO IDs, the three apparently redundant strains included: Dorea longicatena 42FAA, Eubacterium rectale 29FAA, and Eubacterium ventriosum 47FAA. These three species were removed and the replicate counts were updated to reflect the removal of these three species. The list of matched KO IDs specific to a single species was next used to manually create a color key, which matches a unique color to each species that had KO IDs not shared by any other species. The color key was then used to create a list of KO IDs and matching colors, black for shared KO IDs and a different color for each species with unique KO IDs. This list was imported in iPath2.0 and used to create a custom map. This created a list of color conflicts. Any color conflicts were resolved as black, since this meant the pathway was not unique to a single bacteria. The exception was a conflict with the only unique KO ID for Bifidobacterium longum (K00129), further investigation found that the conflict only affected one of the six pathways that the KO ID mapped to and the conflict was resolved not resolved as black but instead matched to specific color for Bifidobacterium longum.

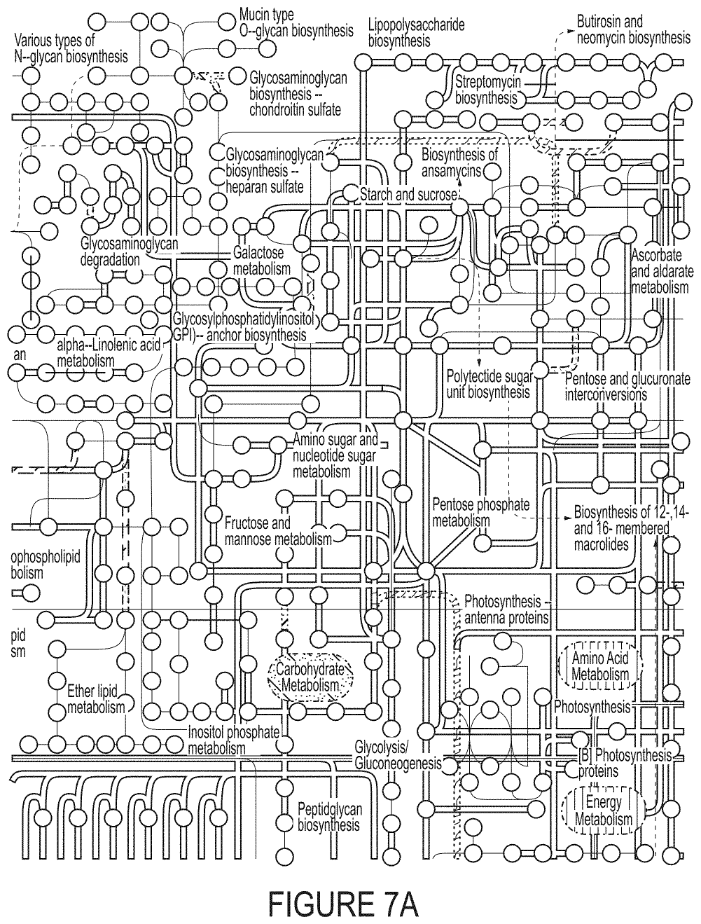

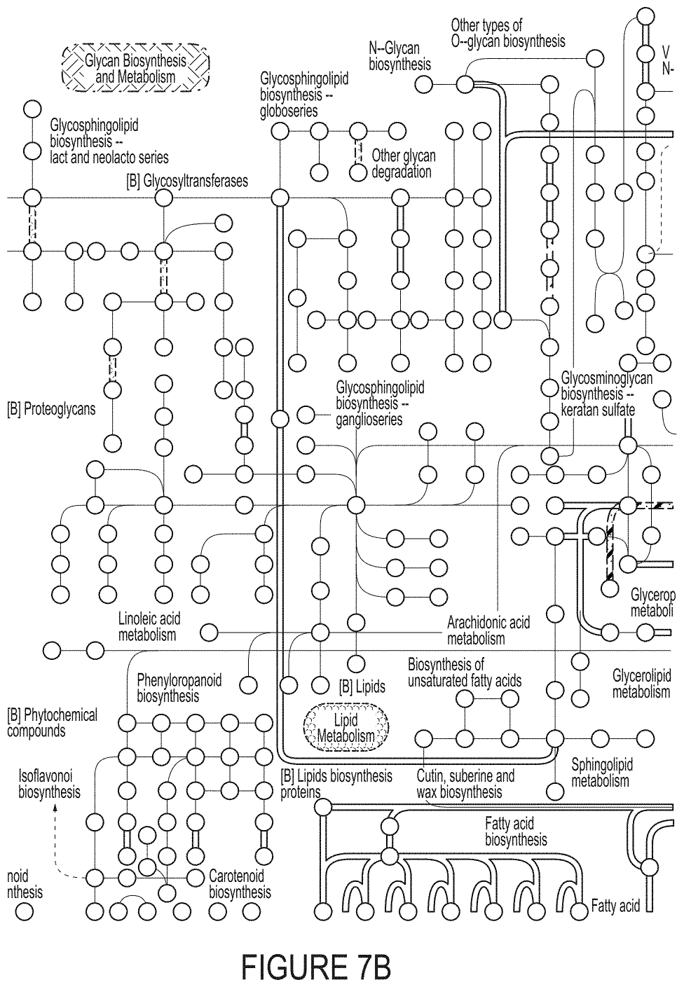

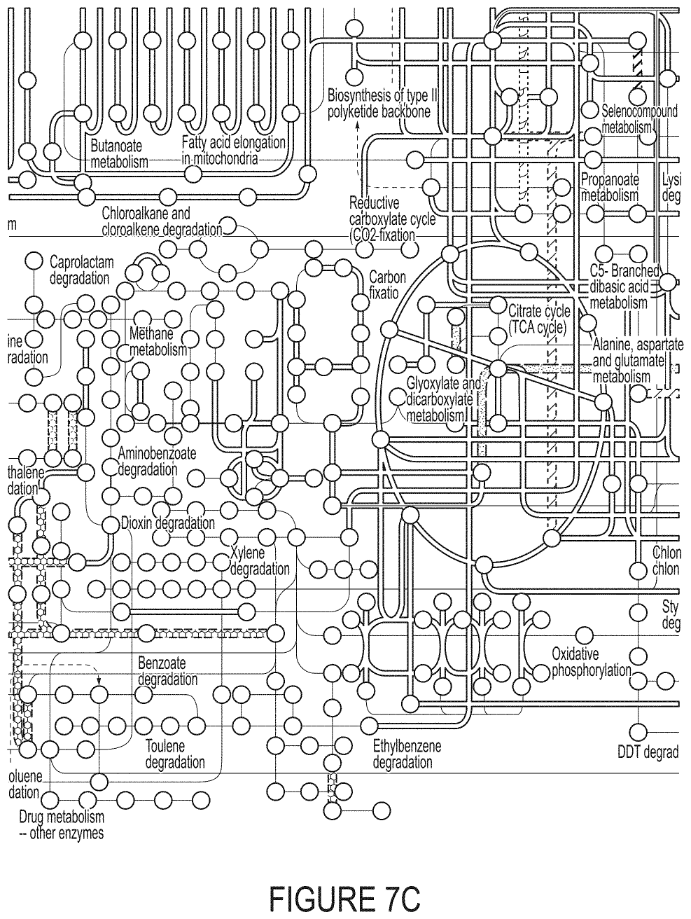

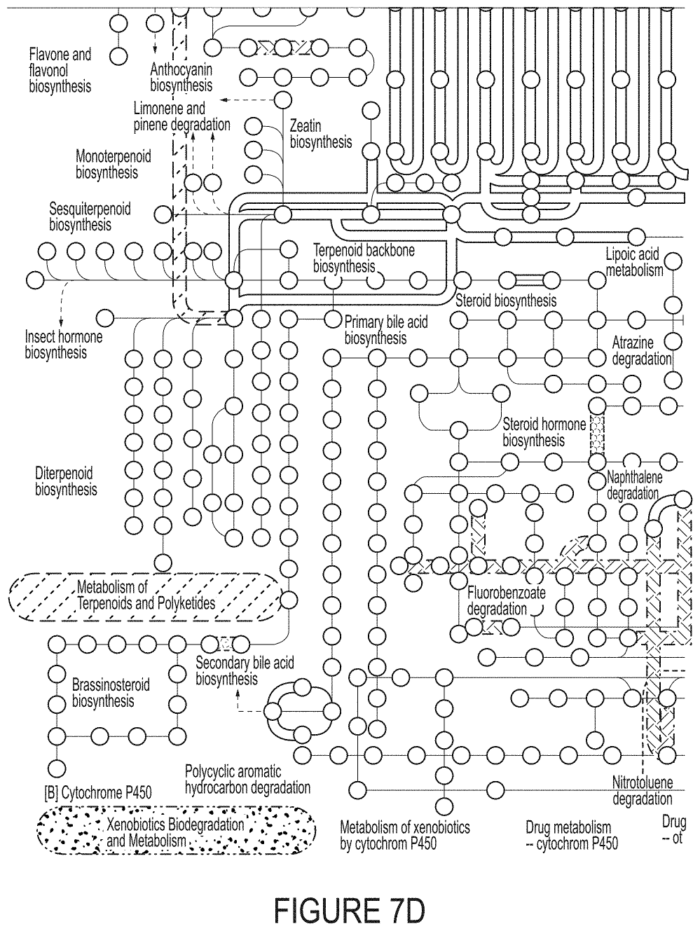

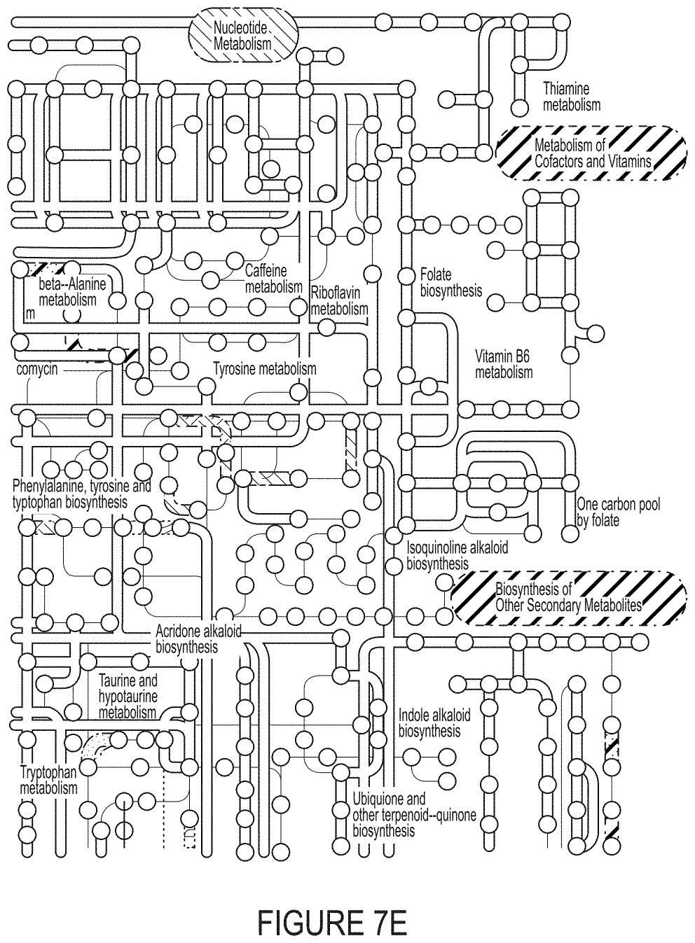

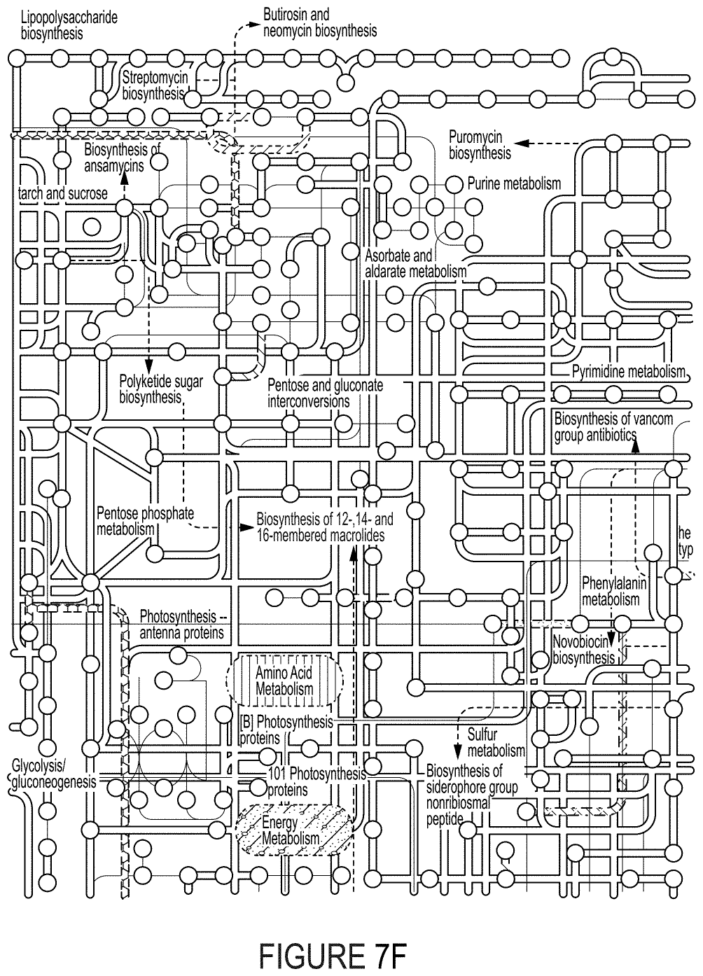

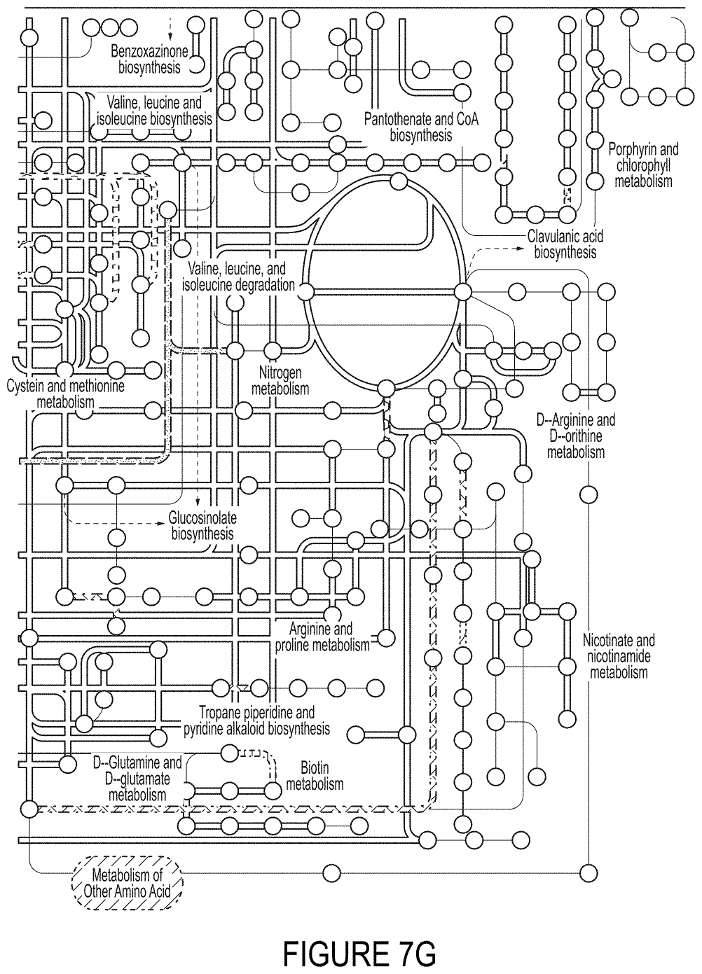

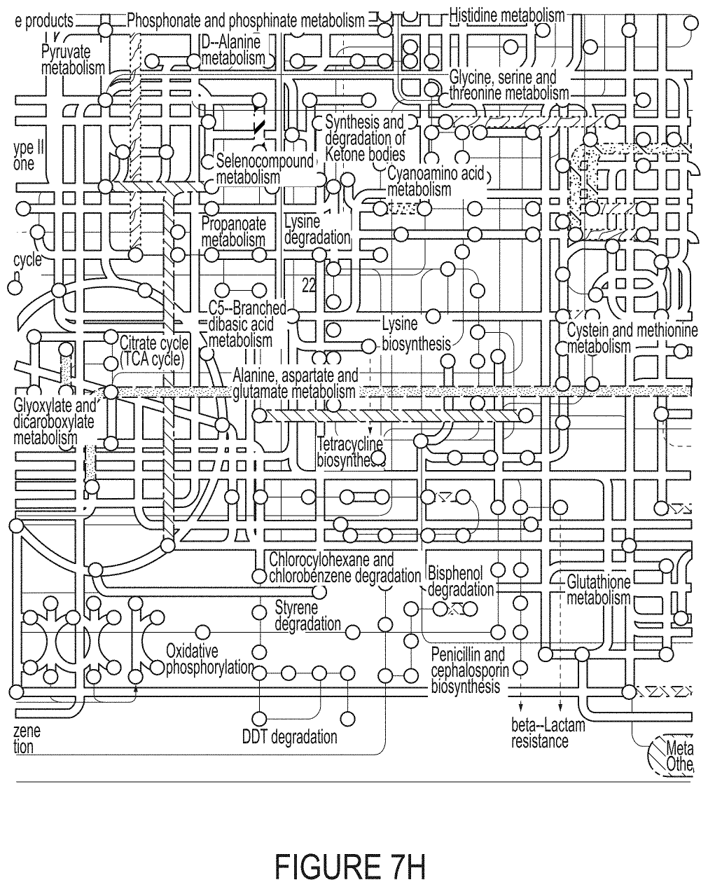

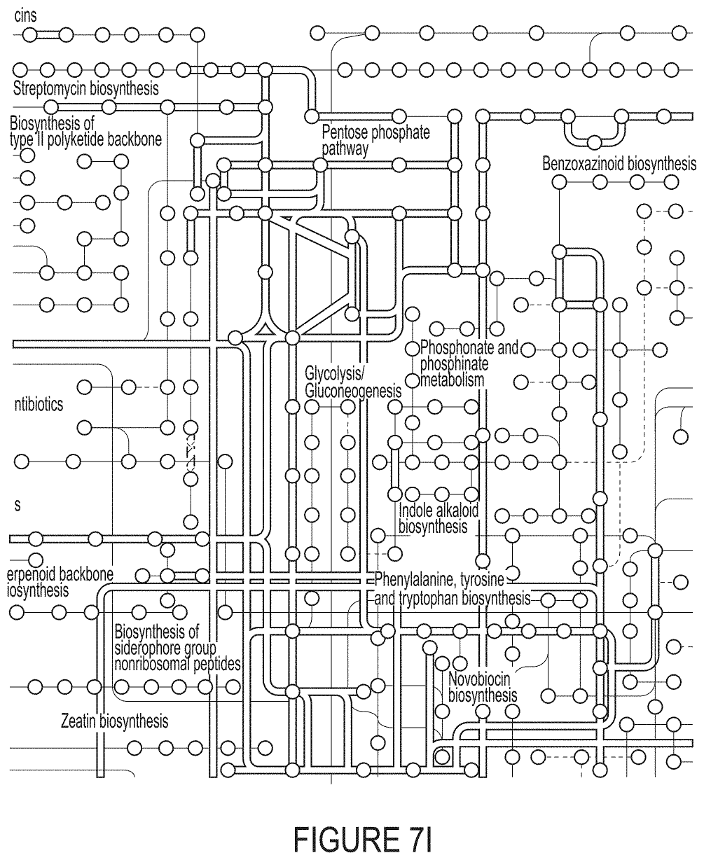

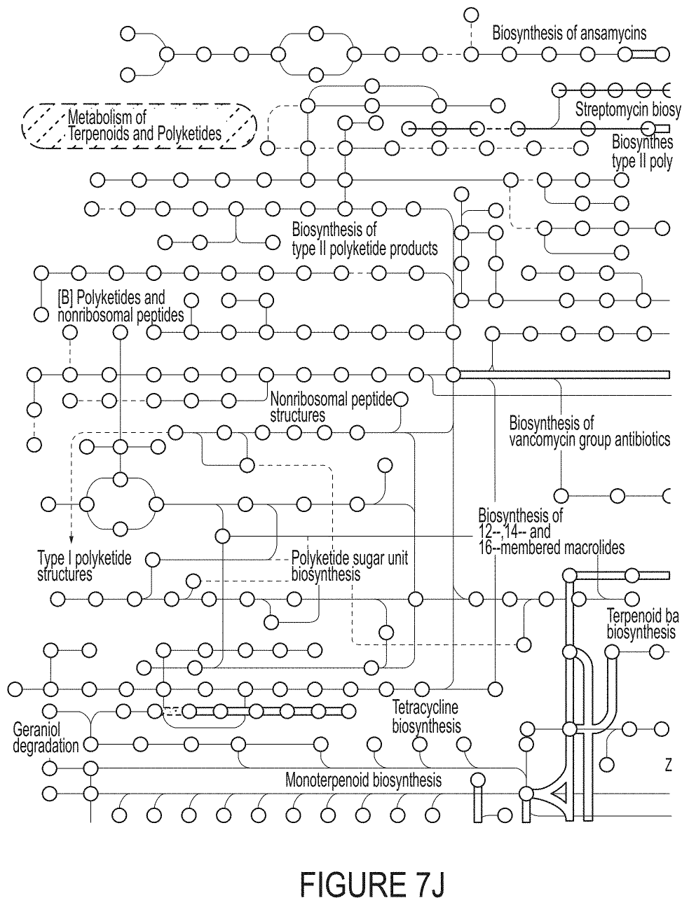

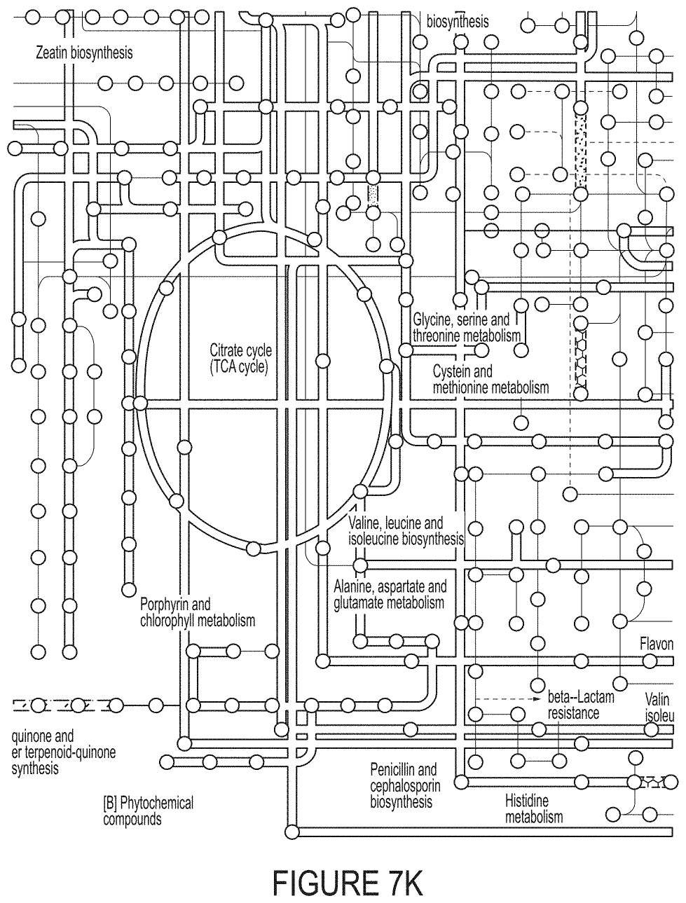

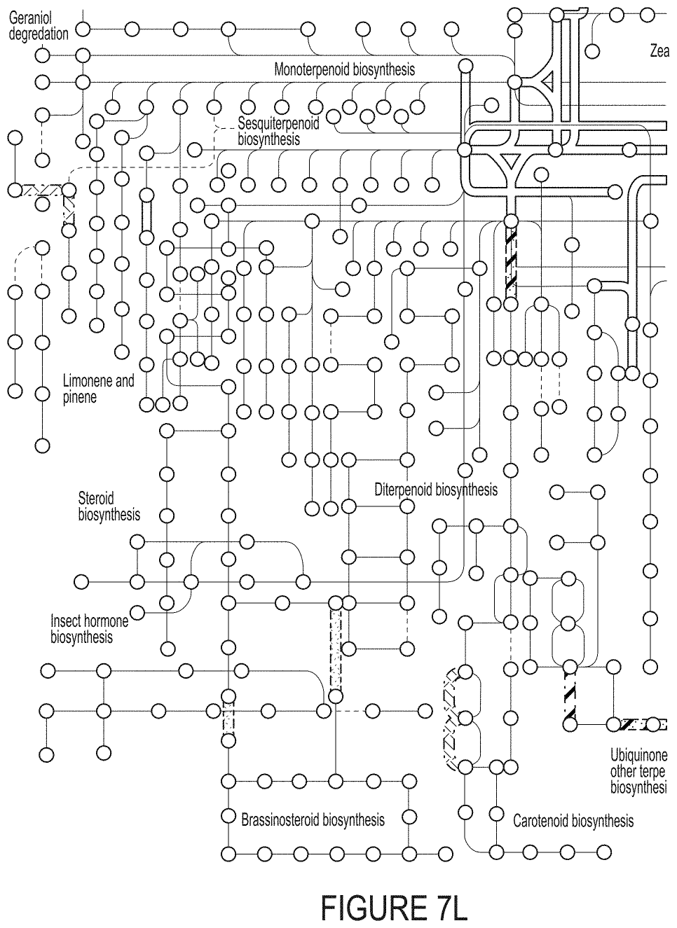

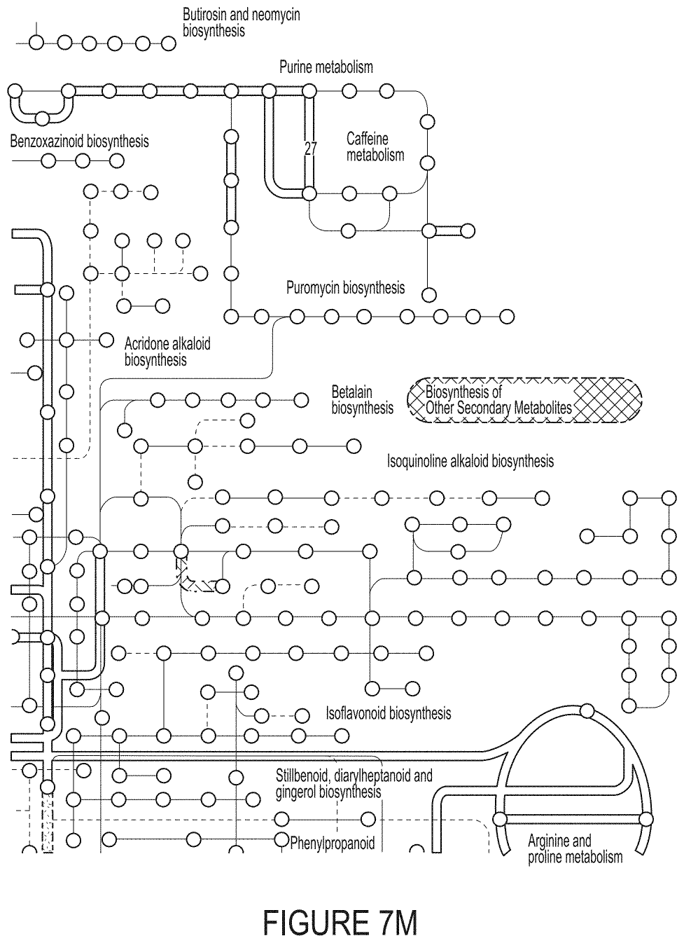

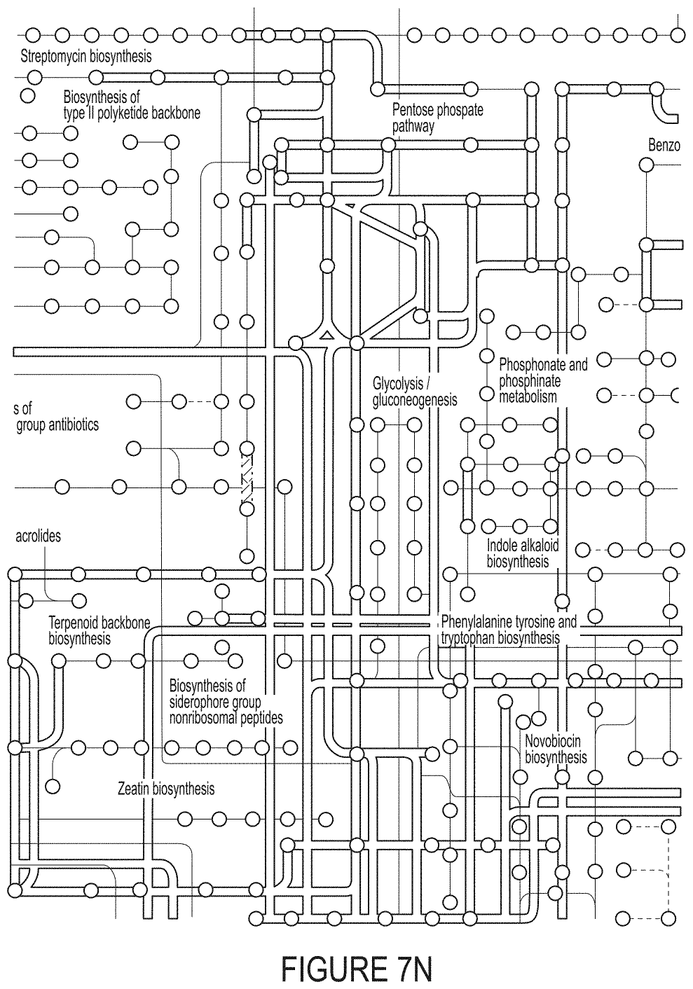

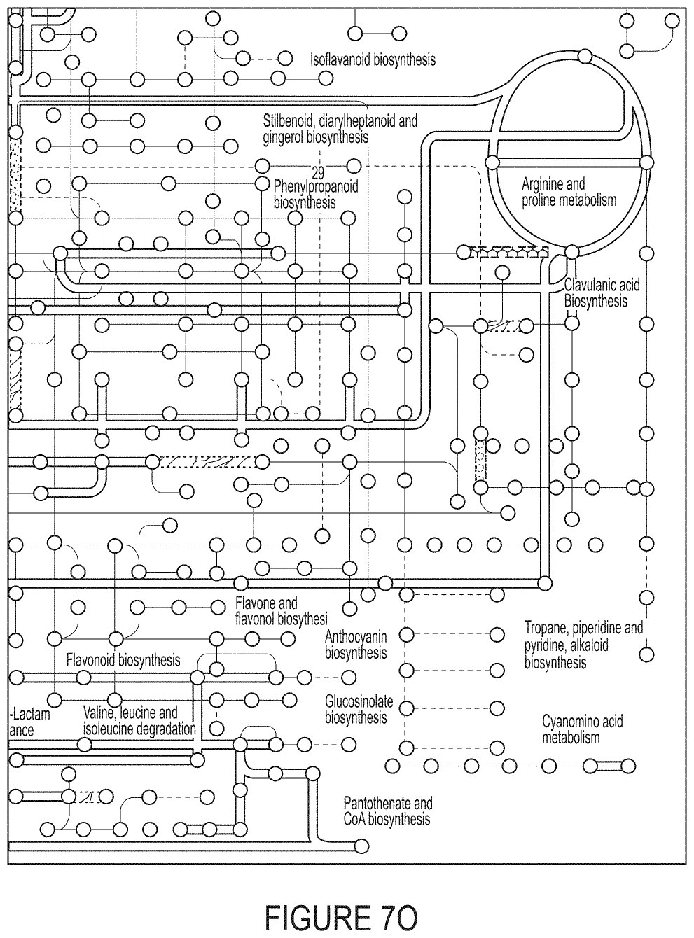

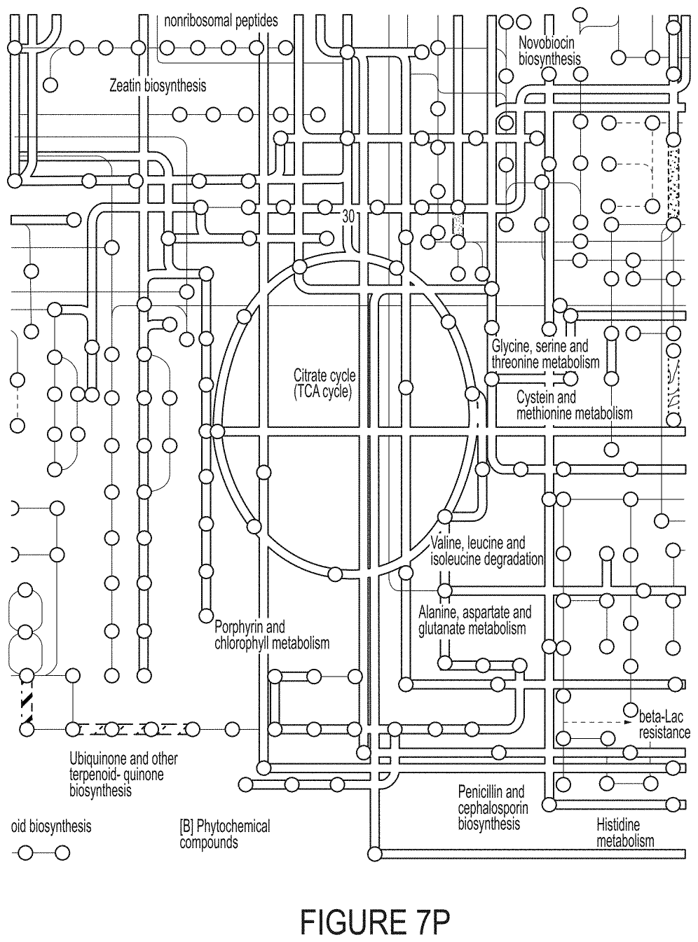

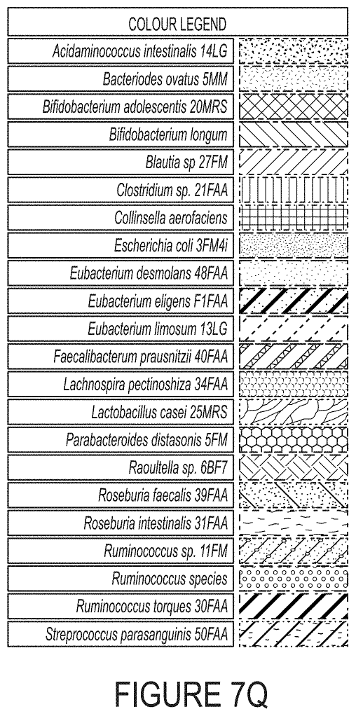

Following conflict resolution a final map was created with black lines for shared pathways and different colored lines for each species with unique KO IDs (FIG. 7). The metabolic and biosynthesis of secondary metabolites maps were analyzed to obtain the number of unique nodes and the highest number of connected nodes. Theses were examined since there are a large number of biochemical and metabolic pathways in bacteria that remain unknown; therefore these element counts may give a better understanding of possible underlying pathways than examining the edges alone (Table 11).

Table 11 shows the element count for ipath2.0 KEGG pathway analysis. A summary of the results for Part II: Redundancy within the RePOOPulate ecosystem including the names of the twenty-two species with unique KO IDs, the number of unique pathway elements that those KO IDs map to for each of the three maps (unique pathways) and a count of the number of unique nodes and the highest number of connected nodes for metabolic and biosynthesis of secondary metabolites maps. Unique nodes were counted if the nodes are part of a unique pathway only and not shared by any other pathways. Numbers in brackets are the number of shared nodes that were also part of a unique pathway. Nodes connected were counted as the highest number of unique nodes connected by unique pathway elements. Numbers in brackets are the highest number of nodes connected by unique pathway elements if the shared nodes that are also part of a unique pathway are included.

FIG. 7 shows the KEGG pathway maps for RePOOPulate population comparison.

FIG. 7A shows a full metabolic pathway map for the comparison of 25 species (redundant strains removed) from the original RePOOPulate ecosystem, showing all pathways unique to a single strain. FIG. 7B shows a full regulatory pathway map for the comparison of all 25 species (redundant strains removed) from the original RePOOPulate ecosystem, showing all pathways unique to a single strain. Color legend to the left indicates which color correlates to which species. Line weights were chosen for ease of visualization and do not reflect the number of copies of the KEGG ID.

TABLE 11

| | | | | Biosynthesis of Secondary | | | |
|---|---|---|---|---|---|---|---|
| | Metabolic | | | Metabolites | | | Regulatory |
| | Unique | Unique | Nodes | Unique | Unique | Nodes | Unique |
| Species | Pathways | Nodes | Connected | Pathways | Nodes | Connected | Pathways |
| Acidaminococcus | 1 | 0 (2) | 0 (2) | 1 | 1 (1) | 1 (2) | 2 |
| intestinalis 14LG | | | | | | | |
| Bacteriodes ovatus | 8 | 12 (4) | 2 | 3 | 5 (1) | 2 | 1 |
| 5MM | | | | | | | |
| Bifidobacterium | 3 | 2 (4) | 1 | 0 | 0 | 0 | 3 |
| adolescentis 20MRS | | | | | | | |
| Bifidobacterium | 4 | 6 (2) | 2 | 0 | 0 | 0 | 0 |
| longum | | | | | | | |
| Blautia sp 27FM | 4 | 4 (4) | 1 (3) | 0 | 0 | 0 | 0 |
| Clostridium sp. | 1 | 1 (1) | 1 (2) | 0 | 0 | 0 | 1 |
| 21FAA | | | | | | | |
| Collinsella | 0 | 0 | 0 | 0 | 0 | 0 | 3 |
| aerofaciens | | | | | | | |
| Escherichia coli | 3 | 3 (3) | 2 (4) | 2 | 1 (1) | 1 (2) | 15 |
| 3FM4i | | | | | | | |
| Eubacterium | 1 | 2 | 2 | 0 | 0 | 0 | 0 |
| desmolans 48FAA | | | | | | | |
| Eubacterium eligens | 4 | 2 (5) | 1 (3) | 0 | 0 | 0 | 0 |
| F1FAA | | | | | | | |
| Eubacterium | 4 | 3 (5) | 2 | 4 | 5 (1) | 4 | 3 |
| limosum 13LG | | | | | | | |
| Faecalibacterium | 0 | 0 | 0 | 0 | 0 | 0 | 1 |
| prausnitzii 40FAA | | | | | | | |
| Lachnospira | 5 | 8 (1) | 2 | 0 | 0 | 0 | 1 |
| pectinoshiza 34FAA | | | | | | | |
| Lactobacillus casei | 7 | 2 (11) | 2 (3) | 1 | 2 | 2 | 1 |
| 25MRS | | | | | | | |
| Parabacteroides | 2 | 1 (3) | 1 | 1 | 1 (1) | 1 (2) | 2 |
| distasonis 5FM | | | | | | | |
| Raoultella sp. 6BF7 | 39 | 46 (14) | 15 (18) | 10 | 16 (2) | 3 | 3 |
| Roseburia faecalis | 0 | 0 | 0 | 0 | 0 | 0 | 2 |
| 39FAA | | | | | | | |
| Roseburia | 3 | 3 (4) | 3 (4) | 0 | 0 | 0 | 2 |
| intestinalis 31FAA | | | | | | | |
| Ruminococcus sp. | 0 | 0 | 0 | 0 | 0 | 0 | 1 |
| 11FM | | | | | | | |
| Ruminococcus | 0 | 0 | 0 | 0 | 0 | 0 | 2 |
| species | | | | | | | |
| Ruminococcus | 2 | 3 (1) | 2 | 1 | 2 | 2 | 1 |
| torques 30FAA | | | | | | | |
| Streprococcus | 3 | 2 (3) | 1 (3) | 0 | 0 | 0 | 4 |
| parasanguinis | | | | | | | |
| 50FAA | | | | | | | |

A final list containing only the unique KO IDs for the twenty-two species with unique KO IDs and matching color codes was used to create maps showing only the unique pathways (FIG. 8). These maps were analyzed to help determine the keystone species and pathways (Table 12). The final list of all KO IDs for the twenty-two species was compared to the list of KO IDs for the original thirty-three species to determine whether any KO IDs had been lost in the process. The list of KO IDs for the final twenty-two species with a list of weights reflecting the number of copies of the KO IDs was used again in Part III of this study. A simple quality check was also performed on the data to see if any obvious errors in the sequencing and genome assembly were evident. Genome size and the number of contigs for all thirty-three genomes were compared using a scatter plot created in R (FIG. 3C). The error in Eubacterium rectale 18FAA, which has been previously noted, was evident and all other genomes appear normal.

Table 12 shows a summary of the unique KEGG pathways of the RePOOPulate ecosystem. Summary of the metabolic and regulatory pathways and the biosynthesis of secondary metabolites for the 22 bacterial species with unique KO IDs after removal of the redundant strains found in Part I. Includes the names of the species with unique KO IDs following matching and conflict resolution with their unique KO IDs and the pathways that they map to. Colors reflect the color legend used for the metabolic and regulatory pathway maps (FIG. 7). KO IDs in red (3) are the unique IDs found only following removal of Dorea longicatena 42FAA, Eubacterium rectale 29FAA, and Eubacterium ventriosum 47FAA in Part II. KO IDs in blue (14) were also found in the Kurokawa et al. data set. Numbers in brackets indicate the number of elements within each of the three maps the KO ID maps to.

FIG. 8 shows the regulatory pathway map for the comparison of twenty-two species from the original RePOOPulate ecosystem (redundant strains removed) showing the regulatory pathways unique to a single strain. Color legend to the left indicates which color correlates to which species. Line weights were chosen for ease of visualization and do not reflect the number of copies of the KO IDs.

TABLE 4

| Included in Optimized Ecosystem | | Removed in Part I |
|---|---|---|
| [symbol] | Faecalibacterium prausnitzii | Bifidobacterium adolescentis |
| | 40FAA | 11FAA |
| [symbol] | Lachnospira pectinoshiza 34FAA | Bifidobacterium longum 4FM |
| [symbol] | Bifidobacterium adolescentis | Dorea longicatena 10FAA |
| | 11FAA | |
| [symbol] | Bifidobacterium longum | Lactobacillus casei 6MRS |
| [symbol] | Blautia sp 27FM | Ruminococcus torques 9FAA |
| [symbol] | Roseburia faecalis 39FAA | Eubacterium rectale |
| [symbol] | Roseburia intestinalis 31FAA | Eubacterium rectale 6FM |
| [symbol] | Ruminococcus species | Eubacterium rectale 18FAA |
| [symbol] | Ruminococcus sp. 11FM | Removed in Part II |
| Collinsella aerofaciens | Ruminococcus torques 30FAA | Dorea longicatena 42FAA |
| Eubacterium desmolans 48FAA | Streprococcus parasanguinis | Eubacterium rectale 29FAA |
| | 50FAA | Eubacterium ventriosum 47FAA |

Table 4. Summary for the RePOOPulate Bacterial Species. Table includes all thirty-three species included in the original RePOOPulate prototype by name listed on the RAST server. Species are separated into three categories based on the analysis in Part I and II. The twenty-two species found to have unique KEGG pathways after removal of the redundant strains found in Part I are in the first two columns, the eight species strains found to be redundant in Part I of the study and three species found to be redundant in Part II are in the last column. The nine species listed in bold are species with unique KO IDs also present in the Kurokawa et al. data, numbers in brackets indicate the number of KO IDs.

Results

The comparison of the unique and almost unique pathways and nodes, shared by one, two, three or four species or strains, revealed several interesting patterns. A comparison of the pathways shared by two, three and four species was done in order to give an idea of redundancy within the ecosystem that cannot be easily removed (because the pathway is rare overall to the ecosystem, but not unique). The KEGG orthology assignment comparison of the twenty-five species within the bacterial community that remained, after the removal of the redundant species in Part I, revealed three species that did not have unique KO IDs and appear to be further redundancies within the ecosystem (Dorea longicatena 42FAA, Eubacterium rectale 29FAA, and Eubacterium ventriosum 47FAA). When the almost unique pathways for these three species were examined there was also only a low number of almost unique pathways. When comparing KO IDs shared by two, three and four species respectively, Eubacterium rectale 29FAA had 3, 1 and 3 shared KO IDs, Dorea longicatena 42FAA had 3, 5 and 3 shared KO IDs and Eubacterium ventriosum 47FAA had 3, 7 and 6 shared KO IDs. This suggests that these three species are not of great importance within the ecosystem and could likely be removed without disrupting the ecological balance.

The comparison of the almost unique KO IDs also revealed the importance of four species that are likely keystone species within the ecosystem. Raoultella sp. 6BF7, Bacteroides ovatus 5MM, Escherichia coli 3FM4i, and Parabacteroides distasonis 5FM all had high levels of almost unique pathway, the majority of which were shared between these four species. Raoultella sp. 6BF7 and Escherichia coli 3FM4i in particular shared an unusually high number of KO IDs when looking at KO ID shared by two species. When examining the KO IDs shared by four species Bacteroides ovatus 5MM and Parabacteroides distasonis 5FM shared a high number of KO IDs with Raoultella sp. 6BF7 and Escherichia coli 3FM4i. This suggests that these four species may interact and play key roles in the ecosystem. Several species were also identified with low levels of almost unique pathways, having three or less KO IDs shared for the comparisons of two, three or four species (Table 5). Faecalibacterium prausnitzii 40FAA, Lachnospira pectinoschiza 34FAA, and Eubacterium rectale 29FAA had low levels of shared KO IDs in all three of the comparisons. Collinsella aerofaciens, and Dorea longicatena 42FAA also had low KO IDs in two of the three comparisons. This suggests that these five species may not play any major role in necessary low-level redundancy.

Table 5 is a summary of a comparison of KEGG orthology assignments shared by two, three or four species. Table 5 summarizes the species found to have low levels of almost unique pathways, having three or less KO IDs shared for between two, three or four species. Species highlighted in bold text fall into this category for two or more comparisons. Numbers in brackets indicate the number of KO IDs shared (prior to conflict resolution).

TABLE 5

| Two | Three Species | Four |
|---|---|---|
| [symbol] | Faecalibacterum prausnitzii | Faecalibacterum prausnitzii |
| | 40FAA (2) | 40FAA (2) |
| [symbol] | Lachnospira pectinoshiza 34FAA | Lachnospira pectinoshiza 34FAA |
| | (3) | (2) |
| [symbol] | Eubacterium rectale 29FAA (1) | Eubacterium rectale 29FAA (3) |
| [symbol] | Collinsella aerofaciens (3) | — |
| [symbol] | — | Dorea longicatena 42FAA (3) |
| Ruminococcus torques 30FAA (3) | Roseburia faecalis 39FAA (1) | — |
| Clostridium sp. 21FAA (3) | Bifidobacterium adolescentis | — |
| | 11FAA (2) | |
| Eubacterium desmolans 48FAA | Roseburia intestinalis 31FAA (3) | — |
| (3) | | |
| Eubacterium ventriosum 47FAA | Eubacterium eligens F1FAA (2) | — |
| (3) | | |

The final pathway analysis resulted in only twenty-two of the thirty-three initial bacteria having unique pathways not covered by any other bacteria within the RePOOPulate system. A list of the final twenty-two species included in the updated model can be found in Table 4. The KEGG pathway map showing the unique pathways for these twenty-two key species can be seen in FIGS. 7 and 8 and a chart listing the pathways that these KO IDs map to can be found in Table 12. The consideration of the number of nodes for each strain that are crossed by pathways unique to the strain allows for a better idea of the possible unique unknown pathways that are present, and by looking at the highest number of connected nodes we gain some idea of the relevance of the pathways, as the higher the number of connected nodes, the higher the likelihood of importance of the pathway. An examination of this data showed, both Bacteroides ovatus 5MM and Lachnospira pectinoschiza 34FAA have a higher numbers of unique nodes than most of the other species (12 and 8 respectively), however the highest number of connected nodes is only 2 for both. This suggests there may be unknown pathways involved. The most relevant species appears to be Raoultella sp. 6BF7, which has 46 unique nodes with the highest number of connected pathways being 15. This is five times greater the species with the next highest number of connected nodes, Roseburia intestinalis 31FAA, which has 3 unique nodes all connected (Table 11).

A comparison of the final list of KO IDs for the twenty-two key species compared to the list of KO IDs for the original thirty-three species revealed a loss of two KO IDs (K07768 and K11695) resulting from the removal of the eight species strains found to be redundant in Part I. The first KO ID was likely lost as a result of the removal of Eubacterium rectale 18FAA. This was the only bacterial species or strain that appeared to have had an error occur in genome assembly, having an overly large number of contigs for a relatively small genome size (FIG. 3C). Further research is required to determine the true importance of this strain. The KO ID that appears to have been lost (K07768) maps to three regulatory pathways within the two-component system for signal transduction, however two of those pathways are also mapped by another KO ID (K07776), which is still present in the final list of KO IDs for the twenty-two species ecosystem. This suggests that only a single small pathway was lost, which would likely not affect the ecological balance. The second KO ID (K11695) lost in the process of redundancy removal maps to a single metabolic pathway for peptidoglycan biosynthesis and is the only KO ID that maps to this pathway. This KO ID was lost as a result of the removal of Bifidobacterium longum 4FM. It is unclear whether the loss of this pathway will have a negative effect on the ecosystem's sustainability and further study is required to determine whether this bacterial strain may be necessary.

A closer look at the unique pathways for the twenty-two species suggests that further optimization of the number of species may be possible. The map showing the unique pathways revealed four bacterial strains with very few unique pathways including: Eubacterium desmolans 48FAA, Faecalibacterium prausnitzii 40FAA, Ruminococcus species (strain A) and Ruminococcus sp. 11FM, each of which only maps to a single map element and only one or two pathways (Table 12). This evidence combined with the information gained from comparing the pathways shared by two, three and four species (Table 5) suggests that Eubacterium desmolans 48FAA and Faecalibacterium prausnitzii 40FAA could likely be removed without causing imbalance in the ecosystem. Lachnospira pectinoschiza 34FAA and Collinsella aerofaciens also showed very few almost unique pathways (Table 5) and only have a few unique KO IDs and pathway elements (Table 12; 3 KO IDS, 6 elements and 2 KO IDs 2 elements, respectively). Further research would be required to determine the necessity of these four species in order to justify their removal or inclusion in a new prototype RePOOPulate ecosystem.

### Part III: Comparison of KEGG Pathway Coverage

### Methods

The list of KO IDs for all thirty-three species with weights determined by number of KO ID replicates within the RePOOPulate ecosystem created in Part II was loaded into ipath2.0 and used to create a custom map with lines colored in blue and weights determined by the number of replicates for each KO ID. Conflicts in weight were resolved using the automatic method used by iPath2.0 of randomly choosing between conflicting weights. The same process was completed for the list of KO IDs and updated weights for the optimized ecosystem consisting of the twenty-two species with unique KO IDs; lines for this map were colored black. The “healthy” human gut microbiome for comparison was taken from a study by Kurokawa et al., which is herein incorporated by reference in its entirety, and a completed list of KO IDs with weights is provided on the iPath web site. The goal of the Kurokawa et al. study was to identify common and variable genomic features of the human gut microbiome. The study comprised of large-scale comparative metagenomic analyses of fecal samples from 13 healthy Japanese individuals of various ages, including unweaned infants. The data from this study had been previous used in the development of iPath2.0 as a demonstration of its capabilities and was chosen for this comparison because of the ease of use under the time limitations. iPath2.0 maps for the Kurokawa et al. data were created using the custom map function and the provided list. The lines for this list are colored red. The custom maps for all three data sets were then downloaded in portable document format (PDF).

The three PDF images were loaded into GIMP 2.8.10 (GNU image manipulationprogram) as separate layers and the transparency was manipulated by coloring to alpha channel such that the Kurokawa et al. data and both sets of RePOOPulate pathways could be visualized. This was done in order to visually compare how well each of the RePOOPulate ecosystems matched an example of the natural human gut microbiome, as well as each other, to determine the coverage of the KEGG pathways. The three lists of KEGG IDs (one for each map), as well as the list of unique KEGG IDs found in Part II were also compared using a Microsoft Excel spreadsheet table. In order to optimize this process the Kurokawa et al. KO IDs were matched to the internal iPath list to remove any KO IDs that did not map to iPath2.0 pathways in the same way that the other lists were matched in Part II.

Results

The matched list of KO IDs for the full thirty-three species RePOOPulate ecosystem was compared to the matched list of Kurokawa et al. KO IDs, which revealed 635 KO IDs found in the RePOOPulate data set, which are not in the Kurokawa et al. data, and 86 KO IDs found in the Kurokawa et al. data but not in RePOOPulate. The two KO IDs removed during the optimization process were not in the Kurokawa et al. data set. Of the KO IDs unique to either the Kurokawa et al. data or RePOOPulate 63 KO IDs had pathways that were shared with unique pathways from the other data set. 27 unique KO IDs for the Kurokawa et al. data had at least one overlapping pathway with the unique KO IDs for RePOOPulate, and 36 unique RePOOPulate KO IDs had at least one pathway shared by the unique KO IDs from the Kurokawa data. Further analysis is required to more closely examine the exact pathways missing from the RePOOPulate ecosystem that should be present in order to maintain a healthy gut microbiome.

The list of KO IDs that were unique to a single species within the twenty-two species of the optimized ecosystem was also compared to the matched Kurokawa et al. data set. Of the 117 unique KO IDs identified only 14 were also in the Kurokawa et al. data, these are highlighted in blue in Table 12. The 14 KO IDs that were unique to a single species and matched the Kurokawa et al. data were found in only nine species, suggesting these species may be the most important in the ecosystem (see Table 4).

A visual comparison of the two RePOOPulate versions with either thirty-three or twenty-two species revealed only small differences in the number of replicates of KO IDs with no obvious loss of data. A visual comparison of the RePOOPulate data and the Kurokawa et al. data revealed some obvious gaps in the number of replicates of a few metabolic pathways in the RePOOPulate data when compared to the Kurokawa et al. data. This is likely do to a much larger number of bacteria present since the majority of these occurrences was in the area metabolism necessary for life, and would therefore be present in all bacterial species and would have a higher number of replicates for a larger variety of species. There are also several areas within the regulatory pathways map that appear to have an under abundance or absence of coverage in the RePOOPulate ecosystem. These include areas of the aminoacyl-tRNA biosynthesis pathways, ABC transporter pathways, two-component system and bacterial secretion system in particular. Further work would be necessary to understand the importance of these missing elements in order to ascertain whether the RePOOPulate system requires further modification to incorporate species that are able to regulate the pathways.

### DISCUSSION

There are several limitations to the study design outlined in this report. One of the major sources of possible error is the high level of manual manipulation of the data sets, which lends itself to the introduction of human error. The methods chosen to resolve conflicts and sort data were not ideal; in the future a more automated, programming-based approach would eliminate many of these possible sources of error and increase the validity of the results.

A second major issue in the design of this study is the general lack of knowledge about the metabolic and biochemical pathways of bacteria. The issue of possible important unknown bacterial pathways lends itself to an inability to correctly identify important species and the misidentification of redundancy. An attempt was made to correct for this error source through an examination of both the nodes and pathways in the analysis, however this does not account for all possible unknowns. Similarly, the use of the program iPath2.0 also introduces a certain element of the unknown since the program does not include all possible pathways or account for all known KEGG orthology assignments. The comparison of KEGG orthology assignments in this project focused solely on those used within the iPath2.0 program, both for simplicity and ease of understanding. However, this meant that of the 4210 KO IDs identified in the thirty-three genomes of the RePOOPulate ecosystem only 1536 were included in comparisons, leaving 2674 KO IDs unexplored in this analysis.

Accordingly, when our understanding improves regarding the metabolic and biochemical pathways of bacteria, this information regarding these pathways will be incorporated into the embodiments of the subject invention.

The analysis outlined in Part II of this report revealed only twenty-two of the thirty-three original strains of bacteria map to unique pathways. This suggests that some or all of these species may be the “keystone” species within the ecosystem and that the other species could possibly be redundant. This analysis does not account for the fact that a certain level of redundancy within the ecosystem may be required, certain bacterial interactions not examined may be ecologically necessary, or unknown bacterial pathways may play a role in the ecological balance of the community. It must also be mentioned that only nine of these species had unique KO IDs also found in the example of a “healthy” microbial community. Further work is required to definitively define the “keystone” species and pathways necessary for balance within the ecosystem of the human gut.

The final comparison in search of redundancies within the RePOOPulate ecosystem was designed to look at a natural “healthy” human gut bacterial population compared to the artificial community of the RePOOPulate project. This proved to be a challenge since a “healthy” bacterial population has yet to be clearly defined. This study data chosen to represent a “healthy” human gut microbiome was chosen because of time limitations; the data was readily available and already in the correct format for the pathway analysis program used in this study. However, the source of data was not ideal since it contained data on only 13 individuals, all of Japanese ancestry, and also included data on unweaned infants, which could be a source of error because of the dynamic nature of the gut microbiome at early stages of development. The fact that all fecal samples were from Japanese individuals could also be a source of error in the data, due to both a lack of diversity across human subjects and the unique diet of the Japanese. Previous studies have shown that the Japanese have a higher abundance of genes derived from marine bacteria do to the high levels of seaweed in the Japanese diet and a requirement for gut bacteria to breakdown this food source. These introduced marine bacterial genes could affect the pathways seen in the data set. If time had allowed a better source of data would have been the Human Microbiome Project or the European initiative MetaHit, which would have provided a source of data more typical of the North American gut microbiome.

### Example: Creation of a Bacterial Community

The next steps in the process of optimizing the RePOOPulate ecosystem involve the actual creation of the suggested bacterial community, in culture, to see if ecological balance is preserved with the removal of the apparently redundant species and strains. The metagenomic approach used in this study cannot tell us whether the identified genes are expressed and at what levels, therefore the actual functional activity of the community should also be examined through a metatranscriptomic approach. Metatranscriptomics uses messenger RNA isolated from the community that has been converted to complementary DNA and sequenced on a high-throughput platform. This approach allows for the characterization gene expression in the microbial ecosystem and would give a greater understanding of the interactions of the community as a whole. Accordingly, upon creating such a bacterial community, the bacterial community will be administered to a patient suffering from a dysbiosis (e.g., but not limited to, IBD, IBS, UC, cancer-related dysbiosis, etc.), and the patient will exhibit an improved gastrointestinal pathology.

### CONCLUSIONS

The evidence outlined in Part I of this study clearly shows redundancy in five of the six species examined. The evidence outlined in Part II is less clear, but there is some indication that several further redundant species can be found within the RePOOPulate ecosystem. The final analysis in Part III indicates that the RePOOPulate community is very close to emulating the metabolic and regulatory pathways of a healthy human gut microbiome. This comparison also indicates that an ecosystem consisting of twenty-two species rather than the original thirty-three would likely result in a more economic artificial bacterial community without loss of functionality or ecological balance. Further study with bacterial culture is required to test this theory.

What is claimed is:

1. A method, wherein the method treats a subject having a dysbiosis, the method comprising:
a. determining a first metabolic profile of the gut microbiome of a subject having a dysbiosis; and
b. changing the first metabolic profile of the gut microbiome of the subject to a second metabolic profile of the gut microbiome of the subject, by administering to the subject a composition comprising at least one bacterial strain selected from the group consisting of Acidaminococcus intestinalis 14LG, Bacteroides ovatus 5MM, Bifidobacterium adolescentis 20MRS, Bifidobacterium longum, Blautia sp. 27FM, Clostridium sp. 21FAA, Collinsella aerofaciens, Escherichia coli 3FM4i, Eubacterium desmolans 48FAA, Eubacterium eligens F1FAA, Eubacterium limosum 13LG, Faecalibacterium prausnitzii 40FAA, Lachnospira pectinoschiza 34FAA, Lactobacillus casei 25MRS, Parabacteroides distasonis 5FM, Roseburia faecalis 39FAA, Roseburia intestinalis 31FAA, Ruminococcus sp. 11FM, Ruminococcus species, and Ruminococcus torques 30FAA,
wherein the composition is administered at a therapeutically effective amount, sufficient to alter the first metabolic profile of the gut microbiome to the second metabolic profile of the gut microbiome,
wherein the first metabolic profile of the gut microbiome is a consequence of the dysbiosis, wherein the second metabolic profile of the gut microbiome treats the subject having the dysbiosis.

2. The method of claim 1, wherein the composition is administered at therapeutically effective amount, sufficient to colonize the gut of the subject.

3. The method of claim 1, wherein the composition comprises at least one bacterial strain selected from the group consisting of: 16-6-I 21 FAA 92% Clostridium cocleatum; 16-6-I 2 MRS 95% Blautia luti; 16-6-I 34 FAA 95% Lachnospira pectinoschiza; 32-6-I 30 D6 FAA 96% Clostridium glycyrrhizinilyticum; and 32-6-I 28 D6 FAA 94% Clostridium lactatifermentans.

4. The method of claim 1, wherein the dysbiosis is associated with gastrointestinal inflammation.

5. The method of claim 4, wherein the gastrointestinal inflammation is a result of at least one disease selected from the group consisting of: inflammatory bowel disease, irritable bowel syndrome, diverticular disease, ulcerative colitis, Crohn's disease, and indeterminate colitis.

6. The method of claim 1, wherein the dysbiosis is a Clostridium difficile infection.

7. The method of claim 1, wherein the dysbiosis is food poisoning.

8. The method of claim 1, wherein the dysbiosis chemotherapy-related dysbiosis.

9. A method, wherein the method treats a subject having a dysbiosis, the method comprising:
a. determining a first metabolic profile of the gut microbiome of a subject having a dysbiosis; and
b. changing the first metabolic profile of the gut microbiome of the subject to a second metabolic profile of the gut microbiome of the subject, by administering to the subject a composition comprising at least one bacterial species selected from the group consisting of Acidaminococcus intestinalis, Bacteroides ovatus, Bifidobacterium adolescentis, Bifidobacterium longum, Blautia sp., Clostridium sp., Collinsella aerofaciens, Escherichia coli, Eubacterium desmolans, Eubacterium eligens, Eubacterium limosum, Faecalibacterium prausnitzii, Lachnospira pectinoschiza, Lactobacillus casei, Parabacteroides distasonis, Roseburia faecalis, Roseburia intestinalis, Ruminococcus sp., Ruminococcus species, and Ruminococcus torques,
wherein the composition is administered at a therapeutically effective amount, sufficient to alter the first metabolic profile of the gut microbiome to the second metabolic profile of the gut microbiome,
wherein the first metabolic profile of the gut microbiome is a consequence of the dysbiosis, wherein the second metabolic profile of the gut microbiome treats the subject having the dysbiosis.

10. The method of claim 9, wherein the composition is administered at therapeutically effective amount, sufficient to colonize the gut of the subject.

11. The method of claim 9, wherein the composition comprises at least one bacterial species selected from the group consisting of: Clostridium cocleatum; Blautia luti; Lachnospira pectinoschiza; Clostridium glycyrrhizinilyticum; and Clostridium lactatifermentans.

12. The method of claim 9, wherein the dysbiosis is associated with gastrointestinal inflammation.

13. The method of claim 12, wherein the gastrointestinal inflammation is a result of at least one disease selected from the group consisting of: inflammatory bowel disease, irritable bowel syndrome, diverticular disease, ulcerative colitis, Crohn's disease, and indeterminate colitis.

14. The method of claim 9, wherein the dysbiosis is a Clostridium difficile infection.

15. The method of claim 9, wherein the dysbiosis is food poisoning.

16. The method of claim 9, wherein the dysbiosis chemotherapy-related dysbiosis.
